(19) United States
(12) Patent Application Publication
(10) Pub. No.: US 2018/0175305 A1
(43) Pub. Date: Jun. 21, 2018

(54) AZAAZENE ANALOGUES AND THEIR USE AS SEMICONDUCTOR

(71) Applicants: Frank WURTHNER, Hochbery (DE); Frank Wurthner, Hochberg (DE); Wan Yue, London (GB); Thomas Gerner, Heidelberg (DE)

(72) Inventors: Frank WURTHNER, Hochbery (DE); Frank Wurthner, Hochberg (DE); Wan Yue, London (GB); Thomas Gerner, Heidelberg (DE)

(21) Appl. No.: 15/128,455

(22) PCT Filed: Mar. 20, 2015
(86) PCT No.: PCT/IB2015/052048

§ 371 Date: Sep. 23, 2016

(51) Int. Cl. H01L 51/00; C07D 471/22; C07D 471/04

(52) U.S. Cl. CPC H01L 51/0072; C07D 471/22; H01L 51/0558; H01L 51/0068; H01L 51/0065; C07D 471/04

(57) ABSTRACT

The present invention provides compounds of formula (1) and an electronic device comprising the compounds as semiconducting material.

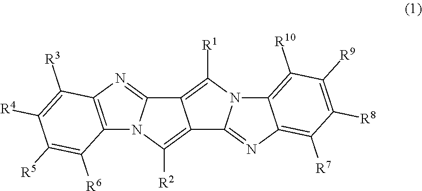

Organic semiconducting materials can be used in electronic devices such as organic photovoltaic devices (OPVs), organic field-effect transistors (OFETs), organic light emitting diodes (OLEDs), and organic electrochromic devices (ECDs).

For efficient and long lasting performance of the electronic device, it is desirable that the organic semiconducting material shows a high chemical stability under ambient air and light conditions.

Furthermore, it is desirable that the organic semiconducting materials are compatible with liquid processing techniques such as spin coating as liquid processing techniques are convenient from the point of processability, and thus allow the production of low cost organic semiconducting material-based electronic devices. In addition, liquid processing techniques are also compatible with plastic substrates, and thus allow the production of light weight and mechanically flexible organic semiconducting material-based electronic devices.

Acenes, other fully-conjugated ring systems and nitrogen-containing analogues thereof have attracted considerable attention in the past years as semiconducting materials for use in electronic devices.

The preparation and characterization of acenes such as pentacenes and hexacenes and their potential as semiconducting material is reviewed by Anthony, J. E. in Angew. Chem. 2008, 120, 460 to 492. Clar, E. Chem. Ber. 1942, 75B, 1330 already pointed out that as the length of acene increases, the stability as well as the solubility significantly decreases. Mondal, R.; Tonshoff, C.; Khon, D.; Neckers, D. C. Bettinger, H. F.; J. Am. Chem. Soc. 2009, 131, 14281 to 14289 showed that hexacene of the following formula

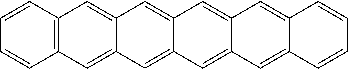 [SMILES: c1ccc2cc3cc4cc5cc6ccccc6cc5cc4cc3cc2c1]

was found to be very unstable in solution.

Chase, D. T.; Fix, A. G.; Kang, S. J.; Rose, B. D.; Weber, C. D.; Zhong, Y.; Zakharow, L. N.; Lonergan, M. C.; Nuckolls, C.; Haley, M. M.; J. Am. Chem. Soc. 2012, 134, 10349-10352 describe the following fully-conjugated ring-system

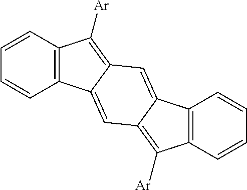

and the use thereof as semiconducting material for organic field effect transistors (OFETs).

Bunz, U. H. F.; Engelhart, J. U.; Lindner, B. D.; Schaffroth, M. Angew. Chem. 2013, 125, 3898-3910 review nitrogen-containing acene derivatives. Miao, S.; Appleton, A. L.; Berger, N.; Barlow, S.; Marder, S. R.; Hardcastle, K. I.; Bunz, U. H. F. describe the following air-stable and soluble tetraazo substituted acene derivatives

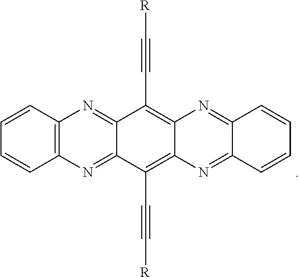

It was the object of the present invention to provide nitrogen-containing fully-conjugated ring systems, which nitrogen-containing fully-conjugated ring systems are of high chemical stability, in particular under ambient temperature, air and light conditions, and are also soluble in organic solvents. In addition, the nitrogen-containing fully-conjugated ring systems should be suitable for use as semiconducting material in electronic devices, in particular in organic field-effect transistors (OFETs).

This object is solved by the compounds of claim 1, the electronic device of claim 8 und the use of claim 10.

The organic semiconducting materials of the present invention are compounds of formula

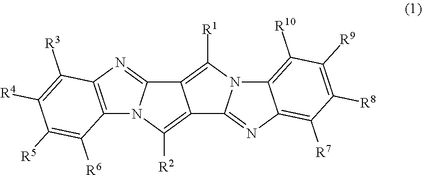

wherein
R¹ and R² are independently from each other selected from the group consisting of H, C₁₋₃₀-alkyl, C₂₋₃₀-alkenyl, C₂₋₃₀-alkynyl, C₅₋₈-cycloalkyl, C₅₋₈-cycloalkenyl, 5 to 14 membered heterocycloalkyl, 5 to 14 membered heterocycloalkenyl, C₆₋₁₄-aryl and 5 to 14 membered heteroaryl, and
R², R³, R⁴, R⁵, R⁶, R⁷, R⁸, R⁹ and R¹⁰ are independently from each other selected from the group consisting of H, C₁₋₃₀-alkyl, C₂₋₃₀-alkenyl, C₂₋₃₀-alkynyl, C₅₋₈-cycloalkyl, C₅₋₈-cycloalkenyl, 5 to 14 membered heterocycloalkyl, 5 to 14 membered heterocycloalkenyl, C₆₋₁₄-aryl, 5 to 14 membered heteroaryl, halogen, CN, —SCN, NO₂, OH, O—C₁₋₃₀-alkyl, O—C₂₋₃₀-alkenyl, O—C₂₋₃₀-alkynyl, O—C₅₋₈-cycloalkyl, O—C₅₋₈-cycloalkenyl, O-5 to 14 membered heterocycloalkyl, O-5 to 14 membered heterocycloalkenyl, O—C₆₋₁₄-aryl, O-5 to 14 membered heteroaryl, SH, S—C₁₋₃₀-alkyl, S—C₂₋₃₀-alkenyl, S—C₂₋₃₀-alkynyl, S—C₅₋₈-cycloalkyl, S—C₅₋₈-cycloalkenyl, S-5 to 14 membered heterocycloalkyl, S-5 to 14 membered heterocycloalkenyl, S—C₆₋₁₄-aryl, S-5 to 14 membered heteroaryl, C(O)H, CO—C₁₋₃₀-alkyl, CO—C₂₋₃₀-alkenyl, CO—C₂₋₃₀-alkynyl, CO—C₅₋₈-cycloalkyl, CO—C₅₋₈-cycloalkenyl, CO-5 to 14 membered heterocycloalkyl, CO-5 to 14 membered heterocycloalkenyl, CO—C₆₋₁₄-aryl, CO-5 to 14 membered heteroaryl, COOH, NH(C₁₋₃₀-alkyl), N(C₁₋₃₀-alkyl)₂, CONH₂, CONH(C₁₋₃₀-alkyl), CON(C₁₋₃₀-alkyl)₂, SO₂OH, SO₂NH₂, SO₂—C₁₋₃₀-alkyl and SO₂—C₆₋₁₄-aryl,
wherein
C₁₋₃₀-alkyl, C₂₋₃₀-alkenyl and C₂₋₃₀-alkynyl, can be substituted with one to nine substituents independently selected from the group consisting of C₅₋₆-cycloalkyl, C₅₋₆-cycloalkenyl, 5 to 10 membered heterocycloalkyl, 5 to 10 membered heterocycloalkenyl, C₆₋₁₀-aryl, 5 to 10 membered heteroaryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂; and one or more CH₂-groups, but not adjacent CH₂-groups of C₁₋₃₀-alkyl, C₂₋₃₀-alkenyl and C₂₋₃₀-alkynyl, and not the CH₂-group directly attached to the core of the compound of formula (1), can be replaced by O or S, and
C₅₋₈-cycloalkyl, C₅₋₈-cycloalkenyl, 5 to 14 membered heterocycloalkyl and 5 to 14 membered heterocycloalkenyl can be substituted with one to five substituents independently selected from the group consisting of C₁₋₂₀-alkyl, C₂₋₂₀-alkenyl, C₂₋₂₀-alkynyl, C₆₋₁₀-aryl, 5 to 10 membered heteroaryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂,
C₆₋₁₄-aryl can be substituted with one to five substituents independently selected from the group consisting of C₁₋₂₀-alkyl, C₂₋₂₀-alkenyl, C₂₋₂₀-alkynyl, C₅₋₆-cycloalkyl, C₅₋₆-cycloalkenyl, 5 to 10 membered heterocycloalkyl, 5 to 10 membered heterocycloalkenyl, and 5 to 10 membered heteroaryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂,
5 to 14 membered heteroaryl can be substituted with one to five substituents independently selected from the group consisting of C₁₋₂₀-alkyl, C₂₋₂₀-alkenyl, C₂₋₂₀-alkynyl, C₅₋₆-cycloalkyl, C₅₋₆-cycloalkenyl, 5 to 10 membered heterocycloalkyl, 5 to 10 membered heterocycloalkenyl, C₆₋₁₀-aryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂,
   wherein
   R^{a} and R^{b} are independently selected from the group consisting of H, C₁₋₂₀-alkyl, C₂₋₂₀-alkenyl and C₂₋₂₀-alkynyl,
   C₁₋₂₀-alkyl, C₂₋₂₀-alkenyl and C₂₋₂₀-alkynyl can be substituted with one to five substituents selected from the group consisting of phenyl, OR^{c}, OC(O)—R^{c}, C(O)—OR^{c}, C(O)—R^{c}, NR^{c}R^{d}, NR^{c}[C(O)R^{d}], N[C(O)R^{c}][C(O)R^{d}], halogen, CN and NO₂, and,
   C₅₋₆-cycloalkyl, C₅₋₆-cycloalkenyl, 5 to 10 membered heterocycloalkyl, 5 to 10 membered heterocycloalkenyl, C₆₋₁₀-aryl and 5 to 10 membered heteroaryl can be substituted with one to five substituents independently selected from the group consisting of C₁₋₁₀-alkyl, C₂₋₁₀-alkenyl, C₂₋₁₀-alkynyl, OR^{c}, OC(O)—R^{c}, C(O)—OR^{c}, C(O)—R^{c}, NR^{c}R^{d}, NR^{c}[C(O)R^{d}], N[C(O)R^{c}][C(O)R^{d}], halogen, CN and NO₂,
      wherein
      R^{c} and R^{d} are independently selected from the group consisting of H, C₁₋₁₀-alkyl, C₂₋₁₀-alkenyl and C₂₋₁₀-alkynyl,
         wherein
         C₁₋₁₀-alkyl, C₂₋₁₀-alkenyl and C₂₋₁₀-alkynyl can be substituted with one to five substituents selected from the group consisting of halogen, CN and NO₂.

Halogen can be F, Cl, Br and I.

C₁₋₁₀-alkyl, C₁₋₂₀-alkyl and C₁₋₃₀-alkyl can be branched or unbranched. Examples of C₁₋₁₀-alkyl are methyl, ethyl, n-propyl, isopropyl, n-butyl, sec-butyl, isobutyl, tert-butyl, n-pentyl, neopentyl, isopentyl, n-(1-ethyl)propyl, n-hexyl, n-heptyl, n-octyl, n-(2-ethyl)hexyl, n-nonyl and n-decyl. Examples of C₁₋₂₀-alkyl are C₁₋₁₀-alkyl and n-undecyl, n-dodecyl, n-undecyl, n-dodecyl, n-tridecyl, n-tetradecyl, n-pentadecyl, n-hexadecyl, n-heptadecyl, n-octadecyl, n-nonadecyl and n-icosyl (C₂₀). Examples of C₁₋₃₀-alkyl are C₁₋₂₀-alkyl and n-docosyl (C₂₂), n-tetracosyl (C₂₄), n-hexacosyl (C₂₆), n-octacosyl (C₂₈) and n-triacontyl (C₃₀).

C₂₋₁₀-alkenyl, C₂₋₂₀-alkenyl and C₂₋₃₀-alkenyl can be branched or unbranched. Examples of C₁₋₂₀-alkenyl are vinyl, propenyl, cis-2-butenyl, trans-2-butenyl, 3-butenyl, cis-2-pentenyl, trans-2-pentenyl, cis-3-pentenyl, trans-3-pentenyl, 4-pentenyl, 2-methyl-3-butenyl, hexenyl, heptenyl, octenyl, nonenyl and docenyl. Examples of C₂₋₂₀-alkenyl are C₂₋₁₀-alkenyl and linoleyl (C₁₈), linolenyl (C₁₈), oleyl (C₁₈), and arachidonyl (C₂₀). Examples of C₂₋₃₀-alkenyl are C₂₋₂₀-alkenyl and erucyl (C₂₂).

C₂₋₁₀-alkynyl, C₂₋₂₀-alkynyl and C₂₋₃₀-alkenyl can be branched or unbranched. Examples of C₂₋₁₀-alkynyl are ethynyl, 2-propynyl, 2-butynyl, 3-butynyl, pentynyl, hexynyl, heptynyl, octynyl, nonynyl and decynyl. Examples of C₂₋₂₀-alkynyl and C₂₋₃₀-alkenyl are undecynyl, dodecynyl, undecynyl, dodecynyl, tridecynyl, tetradecynyl, pentadecynyl, hexadecynyl, heptadecynyl, octadecynyl, nonadecynyl and icosynyl (C₂₀).

Examples of C₆₋₁₀-aryl are phenyl, naphthyl, anthracenyl and phenantrenyl.

Examples of C₆₋₁₄-aryl are C₆₋₁₀-aryl and tetracenyl and chrysenyl.

Examples of C₅₋₈-cycloalkyl are cyclopentyl, cyclohexyl, cycloheptyl and cyclooctyl. Examples of C₅₋₆-cycloalkyl are cyclopentyl and cyclohexyl.

Examples of C₅₋₈-cycloalkenyl are cyclopentenyl, cyclohexenyl, cycloheptenyl and cyclooctenyl.

Examples of C₅₋₆-cycloalkyl are cyclopentenyl and cyclohexenyl.

Examples of 5 to 10 membered heterocycloalkyl and 5 to 14 membered heterocycloalkyl are

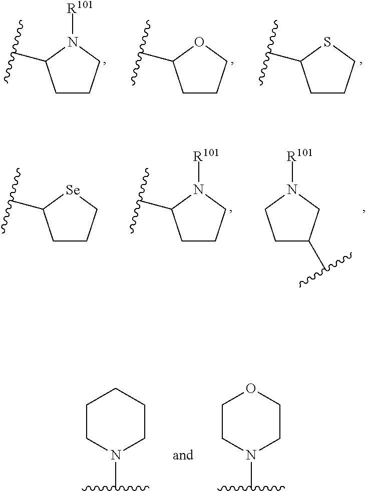

wherein R¹⁰¹ is at each occurrence C₁₋₆-alkyl or phenyl.

Examples of 5 to 10 membered heterocycloalkenyl and 5 to 14 membered heterocycloalkenyl are

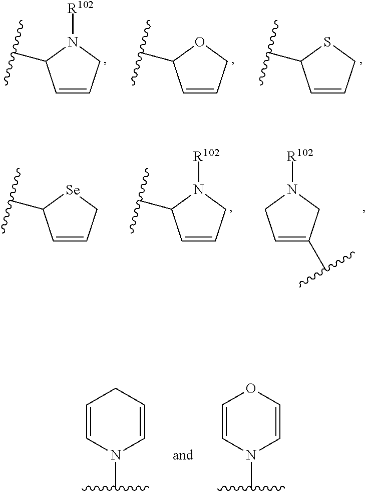

wherein R¹⁰² is at each occurrence C₁₋₆-alkyl or phenyl.

Examples of 5 to 10 membered heteroaryl are

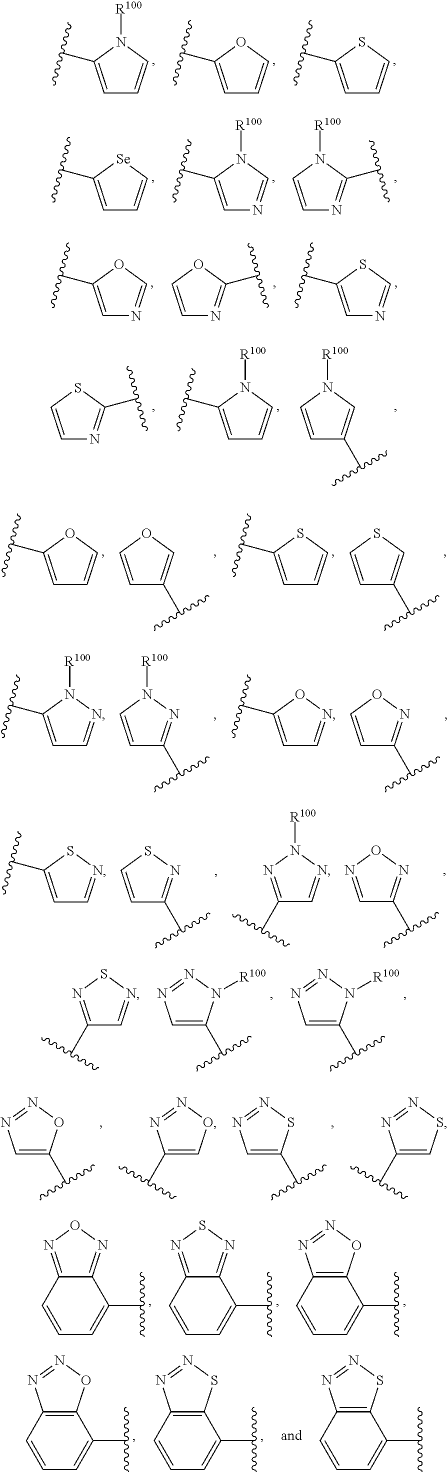

wherein R¹⁰⁰ is at each occurrence C₁₋₆-alkyl or phenyl.

Examples of 5 to 14 membered heteroaryl are the examples given for the 5 to 10 membered heteroaryl and

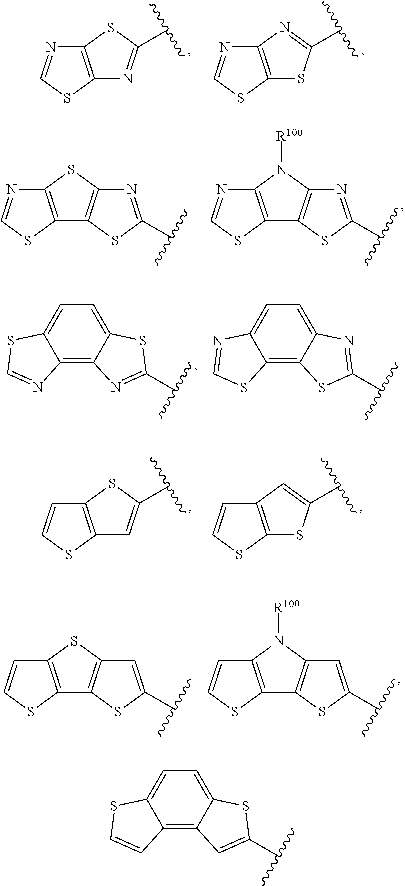

wherein R¹⁰⁰ is at each occurrence C₁₋₆-alkyl or phenyl.

Preferred are compounds of formula

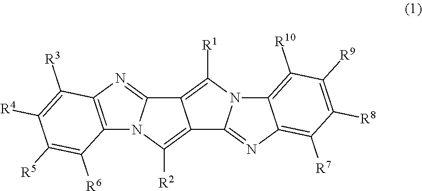

wherein
R¹ and R² are independently from each other selected from the group consisting of H, C₁₋₃₀-alkyl, C₆₋₁₄-aryl, and 5 to 14 membered heteroaryl, and
R², R³, R⁴, R⁵, R⁶, R⁷, R⁸, R⁹ and R¹⁰ are independently from each other selected from the group consisting of H, C₁₋₃₀-alkyl, C₆₋₁₄-aryl, 5 to 14 membered heteroaryl, halogen, CN, —SCN, NO₂, OH, O—C₁₋₃₀-alkyl, O—C₆₋₁₄-aryl, O-5 to 14 membered heteroaryl, SH, S—C₁₋₃₀-alkyl, S—C₆₋₁₄-aryl, S-5 to 14 membered heteroaryl, C(O)H, CO—C₁₋₃₀-alkyl, CO—C₆₋₁₄-aryl, CO-5 to 14 membered heteroaryl, COOH, NH(C₁₋₃₀-alkyl), N(C₁₋₃₀-alkyl)₂, CONH₂, CONH(C₁₋₃₀-alkyl), CON(C₁₋₃₀-alkyl)₂, SO₂OH, SO₂NH₂, SO₂—C₁₋₃₀-alkyl and SO₂—C₆₋₁₄-aryl,
wherein
C₁₋₃₀-alkyl can be substituted with one to nine substituents independently selected from the group consisting of C₆₋₁₀-aryl, 5 to 10 membered heteroaryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂; and one or more CH₂-groups, but not adjacent CH₂-groups of C₁₋₃₀-alkyl and not the CH₂-group directly attached to the core of the compound of formula (1), can be replaced by O or S, and
C₆₋₁₄-aryl can be substituted with one to five substituents independently selected from the group consisting of C₁₋₂₀-alkyl, 5 to 10 membered heteroaryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂,
5 to 14 membered heteroaryl can be substituted with one to five substituents independently selected from the group consisting of C₁₋₂₀-alkyl, C₆₋₁₀-aryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂,
   wherein
   R^{a} and R^{b} are independently selected from the group consisting of H and C₁₋₂₀-alkyl,
   C₁₋₂₀-alkyl can be substituted with one to five substituents selected from the group consisting of phenyl, OR^{c}, OC(O)—R^{c}, C(O)—OR^{c}, C(O)—R^{c}, NR^{c}R^{d}, NR^{c}[C(O)R^{d}], N[C(O)R^{c}][C(O)R^{d}], halogen, CN and NO₂, and,
   C₆₋₁₀-aryl and 5 to 10 membered heteroaryl can be substituted with one to five substituents independently selected from the group consisting of C₁₋₁₀-alkyl, OR^{c}, OC(O)—R^{c}, C(O)—OR^{c}, C(O)—R^{c}, NR^{c}R^{d}, NR^{c}[C(O)R^{d}], N[C(O)R^{c}][C(O)R^{d}], halogen, CN and NO₂,
      wherein
      R^{c} and R^{d} are independently selected from the group consisting of H and C₁₋₁₀-alkyl,
         wherein
         C₁₋₁₀-alkyl can be substituted with one to five substituents selected from the group consisting of halogen, CN and NO₂.

More preferred are compounds of formula

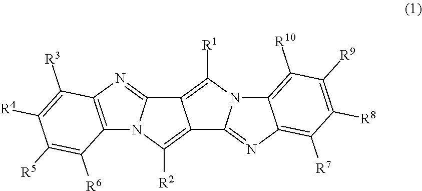

wherein
R¹ and R² are independently from each other selected from the group consisting of C₆₋₁₄-aryl, and 5 to 14 membered heteroaryl, and
R², R³, R⁴, R⁵, R⁶, R⁷, R⁸, R⁹ and R¹⁰ are independently from each other selected from the group consisting of H and C₁₋₃₀-alkyl,
wherein
C₁₋₃₀-alkyl can be substituted with one to nine substituents independently selected from the group consisting of C₆₋₁₀-aryl, 5 to 10 membered heteroaryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂; and one or more CH₂-groups, but not adjacent CH₂-groups of C₁₋₃₀-alkyl and not the CH₂-group directly attached to the core of the compound of formula (1), can be replaced by O or S, and
C₆₋₁₄-aryl can be substituted with one to five substituents independently selected from the group consisting of C₁₋₂₀-alkyl, 5 to 10 membered heteroaryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂,
5 to 14 membered heteroaryl can be substituted with one to five substituents independently selected from the group consisting of C₁₋₂₀-alkyl, C₆₋₁₀-aryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂,
   wherein
   R^{a} and R^{b} are independently selected from the group consisting of H and C₁₋₂₀-alkyl,
   C₁₋₂₀-alkyl can be substituted with one to five substituents selected from the group consisting of phenyl, OR^{c}, OC(O)—R^{c}, C(O)—OR^{c}, C(O)—R^{c}, NR^{c}R^{d}, NR^{c}[C(O)R^{d}], N[C(O)R^{c}][C(O)R^{d}], halogen, CN and NO₂, and,
   C₆₋₁₀-aryl and 5 to 10 membered heteroaryl can be substituted with one to five substituents independently selected from the group consisting of C₁₋₁₀-alkyl, OR^{c}, OC(O)—R^{c}, C(O)—OR^{c}, C(O)—R^{c}, NR^{c}R^{d}, NR^{c}[C(O)R^{d}], N[C(O)R^{c}][C(O)R^{d}], halogen, CN and NO₂,
      wherein
      R^{c} and R^{d} are independently selected from the group consisting of H and C₁₋₁₀-alkyl,
         wherein
         C₁₋₁₀-alkyl can be substituted with one to five substituents selected from the group consisting of halogen, CN and NO₂.

Even more preferred are compounds of formula

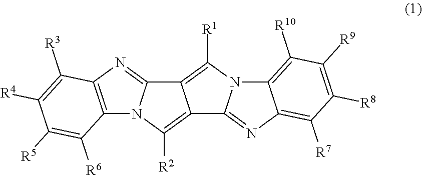

wherein
R¹ and R² are independently from each other selected from the group consisting of C₆₋₁₄-aryl, and 5 to 14 membered heteroaryl, and
R², R³, R⁴, R⁵, R⁶, R⁷, R⁸, R⁹ and R¹⁰ are independently from each other selected from the group consisting of H and C₁₋₃₀-alkyl,
wherein
C₁₋₃₀-alkyl can be substituted with one to nine substituents independently selected from halogen, preferably F, and
C₆₋₁₄-aryl can be substituted with one to five substituents independently selected from C₁₋₂₀-alkyl.

Most preferred are compounds of formula

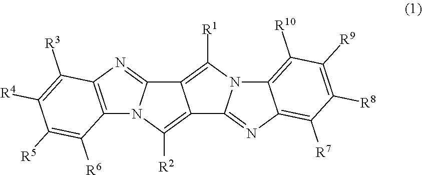

wherein
R¹ and R² are independently from each other selected from the group consisting of phenyl,

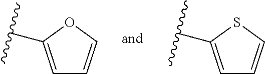 [SMILES: *c1ccco1 ; *c1cccs1]

which can be substituted with one or two substituents independently selected from C₁₋₂₀-alkyl,
and
R², R³, R⁴, R⁵, R⁶, R⁷, R⁸, R⁹ and R¹⁰ are independently from each other selected from the group consisting of H and CF₃.

In particular preferred are the following compounds

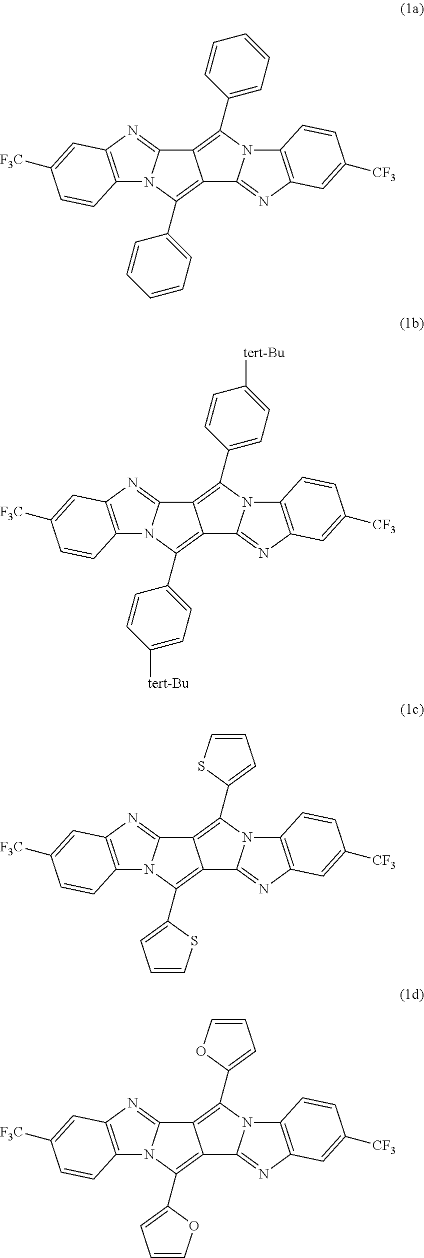

Also part of the invention is a process for the preparation of the compounds of formula 1

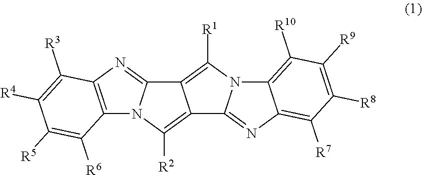

wherein
R¹ and R² are independently from each other selected from the group consisting of H, C₁₋₃₀-alkyl, C₂₋₃₀-alkenyl, C₂₋₃₀-alkynyl, C₅₋₈-cycloalkyl, C₅₋₈-cycloalkenyl, 5 to 14 membered heterocycloalkyl, 5 to 14 membered heterocycloalkenyl, C₆₋₁₄-aryl and 5 to 14 membered heteroaryl, and
R², R³, R⁴, R⁵, R⁶, R⁷, R⁸, R⁹ and R¹⁰ are independently from each other selected from the group consisting of H, C₁₋₃₀-alkyl, C₂₋₃₀-alkenyl, C₂₋₃₀-alkynyl, C₅₋₈-cycloalkyl, C₅₋₈-cycloalkenyl, 5 to 14 membered heterocycloalkyl, 5 to 14 membered heterocycloalkenyl, C₆₋₁₄-aryl, 5 to 14 membered heteroaryl, halogen, CN, —SCN, NO₂, OH, O—C₁₋₃₀-alkyl, O—C₂₋₃₀-alkenyl, O—C₂₋₃₀-alkynyl, O—C₅₋₈-cycloalkyl, O—C₅₋₈-cycloalkenyl, O-5 to 14 membered heterocycloalkyl, O-5 to 14 membered heterocycloalkenyl, O—C₆₋₁₄-aryl, O-5 to 14 membered heteroaryl, SH, S—C₁₋₃₀-alkyl, S—C₂₋₃₀-alkenyl, S—C₂₋₃₀-alkynyl, S—C₅₋₈-cycloalkyl, S—C₅₋₈-cycloalkenyl, S-5 to 14 membered heterocycloalkyl, S-5 to 14 membered heterocycloalkenyl, S—C₆₋₁₄-aryl, S-5 to 14 membered heteroaryl, C(O)H, CO—C₁₋₃₀-alkyl, CO—C₂₋₃₀-alkenyl, CO—C₂₋₃₀-alkynyl, CO—C₅₋₈-cycloalkyl, CO—C₅₋₈-cycloalkenyl, CO-5 to 14 membered heterocycloalkyl, CO-5 to 14 membered heterocycloalkenyl, CO—C₆₋₁₄-aryl, CO-5 to 14 membered heteroaryl, COOH, NH(C₁₋₃₀-alkyl), N(C₁₋₃₀-alkyl)₂, CONH₂, CONH(C₁₋₃₀-alkyl), CON(C₁₋₃₀-alkyl)₂, SO₂OH, SO₂NH₂, SO₂—C₁₋₃₀-alkyl and SO₂—C₆₋₁₄-aryl,
wherein
C₁₋₃₀-alkyl, C₂₋₃₀-alkenyl and C₂₋₃₀-alkynyl, can be substituted with one to nine substituents independently selected from the group consisting of C₅₋₆-cycloalkyl, C₅₋₆-cycloalkenyl, 5 to 10 membered heterocycloalkyl, 5 to 10 membered heterocycloalkenyl, C₆₋₁₀-aryl, 5 to 10 membered heteroaryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂; and one or more CH₂-groups, but not adjacent CH₂-groups of C₁₋₃₀-alkyl, C₂₋₃₀-alkenyl and C₂₋₃₀-alkynyl, and not the CH₂-group directly attached to the core of the compound of formula (1), can be replaced by O or S, and
C₅₋₈-cycloalkyl, C₅₋₈-cycloalkenyl, 5 to 14 membered heterocycloalkyl and 5 to 14 membered heterocycloalkenyl can be substituted with one to five substituents independently selected from the group consisting of C₁₋₂₀-alkyl, C₂₋₂₀-alkenyl, C₂₋₂₀-alkynyl, C₆₋₁₀-aryl, 5 to 10 membered heteroaryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂,
C₆₋₁₄-aryl can be substituted with one to five substituents independently selected from the group consisting of C₁₋₂₀-alkyl, C₂₋₂₀-alkenyl, C₂₋₂₀-alkynyl, C₅₋₆-cycloalkyl, C₅₋₆-cycloalkenyl, 5 to 10 membered heterocycloalkyl, 5 to 10 membered heterocycloalkenyl, and 5 to 10 membered heteroaryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂,
5 to 14 membered heteroaryl can be substituted with one to five substituents independently selected from the group consisting of C₁₋₂₀-alkyl, C₂₋₂₀-alkenyl, C₂₋₂₀-alkynyl, C₅₋₆-cycloalkyl, C₅₋₆-cycloalkenyl, 5 to 10 membered heterocycloalkyl, 5 to 10 membered heterocycloalkenyl, C₆₋₁₀-aryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂,
   wherein
   R^{a} and R^{b} are independently selected from the group consisting of H, C₁₋₂₀-alkyl, C₂₋₂₀-alkenyl and C₂₋₂₀-alkynyl,
   C₁₋₂₀-alkyl, C₂₋₂₀-alkenyl and C₂₋₂₀-alkynyl can be substituted with one to five substituents selected from the group consisting of phenyl, OR^{c}, OC(O)—R^{c}, C(O)—OR^{c}, C(O)—R^{c}, NR^{c}R^{d}, NR^{c}[C(O)R^{d}], N[C(O)R^{c}][C(O)R^{d}], halogen, CN and NO₂, and,
   C₅₋₆-cycloalkyl, C₅₋₆-cycloalkenyl, 5 to 10 membered heterocycloalkyl, 5 to 10 membered heterocycloalkenyl, C₆₋₁₀-aryl and 5 to 10 membered heteroaryl can be substituted with one to five substituents independently selected from the group consisting of C₁₋₁₀-alkyl, C₂₋₁₀-alkenyl, C₂₋₁₀-alkynyl, OR^{c}, OC(O)—R^{c}, C(O)—OR^{c}, C(O)—R^{c}, NR^{c}R^{d}, NR^{c}[C(O)R^{d}], N[C(O)R^{c}][C(O)R^{d}], halogen, CN and NO₂,
      wherein
      R^{c} and R^{d} are independently selected from the group consisting of H, C₁₋₁₀-alkyl, C₂₋₁₀-alkenyl and C₂₋₁₀-alkynyl,
         wherein
         C₁₋₁₀-alkyl, C₂₋₁₀-alkenyl and C₂₋₁₀-alkynyl can be substituted with one to five substituents selected from the group consisting of halogen, CN and NO₂,
            which process comprises the steps of
i) reducing a compound of formula 2

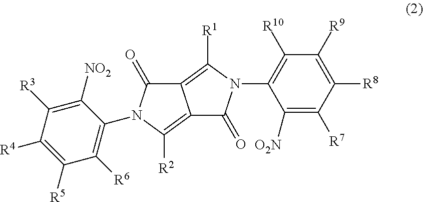

wherein R¹, R², R³, R⁴, R⁵, R⁶, R⁷, R⁸, R⁹ and R¹⁰ are as defined for the compound of formula 1, to the compound of formula 2′

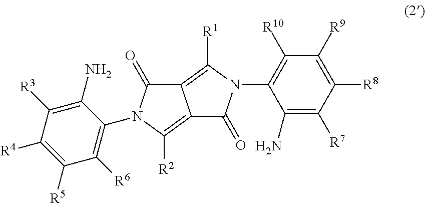

wherein R¹, R², R³, R⁴, R⁵, R⁶, R⁷, R⁸, R⁹ and R¹⁰ are as defined for the compound of formula 1,
and
ii) treating the compound of formula 2′ with a suitable catalyst to obtain a compound of formula 1.

Preferably, the first step includes treating the compound of formula 2 with a suitable catalyst such as SnCl₂ in the presence of a suitable solvent such as ethyl acetate. Preferably, the first step is carried out at elevated temperatures, such as at temperatures from 50 to 150° C., preferably 60 to 100° C.

Preferably, the suitable catalyst of the second step is titanium tetrachloride. Preferably, the second step includes treating compound 2′ with titanium tetrachloride as catalyst, and a suitable base such as DABCO in a suitable solvent such as mesitylene. Preferably, the second step is carried out at elevated temperatures, such as at temperatures from 80 to 180° C., preferably 100 to 150° C.

The compound of formula 2

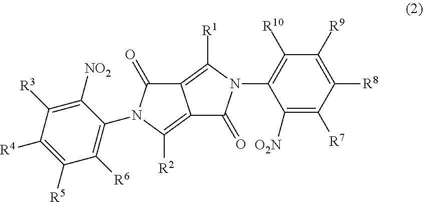

wherein
R¹ and R² are independently from each other selected from the group consisting of H, C₁₋₃₀-alkyl, C₂₋₃₀-alkenyl, C₂₋₃₀-alkynyl, C₅₋₈-cycloalkyl, C₅₋₈-cycloalkenyl, 5 to 14 membered heterocycloalkyl, 5 to 14 membered heterocycloalkenyl, C₆₋₁₄-aryl and 5 to 14 membered heteroaryl, and
R², R³, R⁴, R⁵, R⁶, R⁷, R⁸, R⁹ and R¹⁰ are independently from each other selected from the group consisting of H, C₁₋₃₀-alkyl, C₂₋₃₀-alkenyl, C₂₋₃₀-alkynyl, C₅₋₈-cycloalkyl, C₅₋₈-cycloalkenyl, 5 to 14 membered heterocycloalkyl, 5 to 14 membered heterocycloalkenyl, C₆₋₁₄-aryl, 5 to 14 membered heteroaryl, halogen, CN, —SCN, NO₂, OH, O—C₁₋₃₀-alkyl, O—C₂₋₃₀-alkenyl, O—C₂₋₃₀-alkynyl, O—C₅₋₈-cycloalkyl, O—C₅₋₈-cycloalkenyl, O-5 to 14 membered heterocycloalkyl, O-5 to 14 membered heterocycloalkenyl, O—C₆₋₁₄-aryl, O-5 to 14 membered heteroaryl, SH, S—C₁₋₃₀-alkyl, S—C₂₋₃₀-alkenyl, S—C₂₋₃₀-alkynyl, S—C₅₋₈-cycloalkyl, S—C₅₋₈-cycloalkenyl, S-5 to 14 membered heterocycloalkyl, S-5 to 14 membered heterocycloalkenyl, S—C₆₋₁₄-aryl, S-5 to 14 membered heteroaryl, C(O)H, CO—C₁₋₃₀-alkyl, CO—C₂₋₃₀-alkenyl, CO—C₂₋₃₀-alkynyl, CO—C₅₋₈-cycloalkyl, CO—C₅₋₈-cycloalkenyl, CO-5 to 14 membered heterocycloalkyl, CO-5 to 14 membered heterocycloalkenyl, CO—C₆₋₁₄-aryl, CO-5 to 14 membered heteroaryl, COOH, NH(C₁₋₃₀-alkyl), N(C₁₋₃₀-alkyl)₂, CONH₂, CONH(C₁₋₃₀-alkyl), CON(C₁₋₃₀-alkyl)₂, SO₂OH, SO₂NH₂, SO₂—C₁₋₃₀-alkyl and SO₂—C₆₋₁₄-aryl,
wherein
C₁₋₃₀-alkyl, C₂₋₃₀-alkenyl and C₂₋₃₀-alkynyl, can be substituted with one to nine substituents independently selected from the group consisting of C₅₋₆-cycloalkyl, C₅₋₆-cycloalkenyl, 5 to 10 membered heterocycloalkyl, 5 to 10 membered heterocycloalkenyl, C₆₋₁₀-aryl, 5 to 10 membered heteroaryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂; and one or more CH₂-groups, but not adjacent CH₂-groups of C₁₋₃₀-alkyl, C₂₋₃₀-alkenyl and C₂₋₃₀-alkynyl, and not the CH₂-group directly attached to the core of the compound of formula (1), can be replaced by O or S, and
C₅₋₈-cycloalkyl, C₅₋₈-cycloalkenyl, 5 to 14 membered heterocycloalkyl and 5 to 14 membered heterocycloalkenyl can be substituted with one to five substituents independently selected from the group consisting of C₁₋₂₀-alkyl, C₂₋₂₀-alkenyl, C₂₋₂₀-alkynyl, C₆₋₁₀-aryl, 5 to 10 membered heteroaryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂,
C₆₋₁₄-aryl can be substituted with one to five substituents independently selected from the group consisting of C₁₋₂₀-alkyl, C₂₋₂₀-alkenyl, C₂₋₂₀-alkynyl, C₅₋₆-cycloalkyl, C₅₋₆-cycloalkenyl, 5 to 10 membered heterocycloalkyl, 5 to 10 membered heterocycloalkenyl, and 5 to 10 membered heteroaryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂,
5 to 14 membered heteroaryl can be substituted with one to five substituents independently selected from the group consisting of C₁₋₂₀-alkyl, C₂₋₂₀-alkenyl, C₂₋₂₀-alkynyl, C₅₋₆-cycloalkyl, C₅₋₆-cycloalkenyl, 5 to 10 membered heterocycloalkyl, 5 to 10 membered heterocycloalkenyl, C₆₋₁₀-aryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂,
   wherein
   R^{a} and R^{b} are independently selected from the group consisting of H, C₁₋₂₀-alkyl, C₂₋₂₀-alkenyl and C₂₋₂₀-alkynyl,
   C₁₋₂₀-alkyl, C₂₋₂₀-alkenyl and C₂₋₂₀-alkynyl can be substituted with one to five substituents selected from the group consisting of phenyl, OR^{c}, OC(O)—R^{c}, C(O)—OR^{c}, C(O)—R^{c}, NR^{c}R^{d}, NR^{c}[C(O)R^{d}], N[C(O)R^{c}][C(O)R^{d}], halogen, CN and NO₂, and,
   C₅₋₆-cycloalkyl, C₅₋₆-cycloalkenyl, 5 to 10 membered heterocycloalkyl, 5 to 10 membered heterocycloalkenyl, C₆₋₁₀-aryl and 5 to 10 membered heteroaryl can be substituted with one to five substituents independently selected from the group consisting of C₁₋₁₀-alkyl, C₂₋₁₀-alkenyl, C₂₋₁₀-alkynyl, OR^{c}, OC(O)—R^{c}, C(O)—OR^{c}, C(O)—R^{c}, NR^{c}R^{d}, NR^{c}[C(O)R^{d}], N[C(O)R^{c}][C(O)R^{d}], halogen, CN and NO₂,
      wherein
      R^{c} and R^{d} are independently selected from the group consisting of H, C₁₋₁₀-alkyl, C₂₋₁₀-alkenyl and C₂₋₁₀-alkynyl,
         wherein
         C₁₋₁₀-alkyl, C₂₋₁₀-alkenyl and C₂₋₁₀-alkynyl can be substituted with one to five substituents selected from the group consisting of halogen, CN and NO₂,
            can be prepared by treating a compound of formula 3

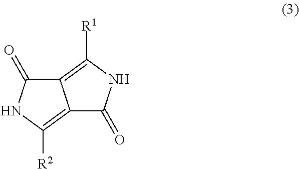

wherein R¹ and R² are as defined for the compound of formula 2,
with compounds of formulae 4 and 4′

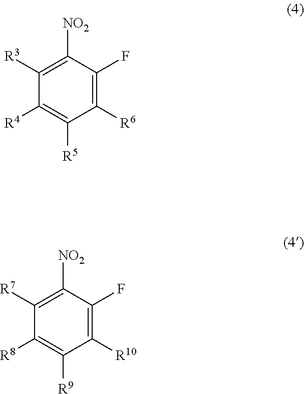

wherein R¹, R², R³, R⁴, R⁵, R⁶, R⁷, R⁸, R⁹ and R¹⁰ are as defined for the compound of formula 2.

Preferably, the reaction is carried out in the presence of a base such as K₂CO₃, and in the presence of a suitable solvent, such as DMF. Preferably, the reaction is carried out at elevated temperatures, such as at temperatures from 50 to 150° C., preferably 60 to 100° C.

The compound of formula 3 can be prepared as described by Potrawa, T.; Langhals, H. Chem. Ber. 1987, 120, 1075-1078, and Woo, C. H.; Beaujuge, P. M.; Holcombe, T. W.; Lee, O. P.; Fréchet, J. M. J. J. Am. Chem. Soc. 2010, 132, 15547-15549.

Also part of the invention are the intermediate compounds of formula 2

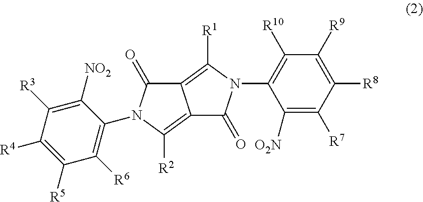

wherein
R¹ and R² are independently from each other selected from the group consisting of H, C₁₋₃₀-alkyl, C₂₋₃₀-alkenyl, C₂₋₃₀-alkynyl, C₅₋₈-cycloalkyl, C₅₋₈-cycloalkenyl, 5 to 14 membered heterocycloalkyl, 5 to 14 membered heterocycloalkenyl, C₆₋₁₄-aryl and 5 to 14 membered heteroaryl, and
R², R³, R⁴, R⁵, R⁶, R⁷, R⁸, R⁹ and R¹⁰ are independently from each other selected from the group consisting of H, C₁₋₃₀-alkyl, C₂₋₃₀-alkenyl, C₂₋₃₀-alkynyl, C₅₋₈-cycloalkyl, C₅₋₈-cycloalkenyl, 5 to 14 membered heterocycloalkyl, 5 to 14 membered heterocycloalkenyl, C₆₋₁₄-aryl, 5 to 14 membered heteroaryl, halogen, CN, —SCN, NO₂, OH, O—C₁₋₃₀-alkyl, O—C₂₋₃₀-alkenyl, O—C₂₋₃₀-alkynyl, O—C₅₋₈-cycloalkyl, O—C₅₋₈-cycloalkenyl, O-5 to 14 membered heterocycloalkyl, O-5 to 14 membered heterocycloalkenyl, O—C₆₋₁₄-aryl, O-5 to 14 membered heteroaryl, SH, S—C₁₋₃₀-alkyl, S—C₂₋₃₀-alkenyl, S—C₂₋₃₀-alkynyl, S—C₅₋₈-cycloalkyl, S—C₅₋₈-cycloalkenyl, S-5 to 14 membered heterocycloalkyl, S-5 to 14 membered heterocycloalkenyl, S—C₆₋₁₄-aryl, S-5 to 14 membered heteroaryl, C(O)H, CO—C₁₋₃₀-alkyl, CO—C₂₋₃₀-alkenyl, CO—C₂₋₃₀-alkynyl, CO—C₅₋₈-cycloalkyl, CO—C₅₋₈-cycloalkenyl, CO-5 to 14 membered heterocycloalkyl, CO-5 to 14 membered heterocycloalkenyl, CO—C₆₋₁₄-aryl, CO-5 to 14 membered heteroaryl, COOH, NH(C₁₋₃₀-alkyl), N(C₁₋₃₀-alkyl)₂, CONH₂, CONH(C₁₋₃₀-alkyl), CON(C₁₋₃₀-alkyl)₂, SO₂OH, SO₂NH₂, SO₂—C₁₋₃₀-alkyl and SO₂—C₆₋₁₄-aryl,
wherein
C₁₋₃₀-alkyl, C₂₋₃₀-alkenyl and C₂₋₃₀-alkynyl, can be substituted with one to nine substituents independently selected from the group consisting of C₅₋₆-cycloalkyl, C₅₋₆-cycloalkenyl, 5 to 10 membered heterocycloalkyl, 5 to 10 membered heterocycloalkenyl, C₆₋₁₀-aryl, 5 to 10 membered heteroaryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂; and one or more CH₂-groups, but not adjacent CH₂-groups of C₁₋₃₀-alkyl, C₂₋₃₀-alkenyl and C₂₋₃₀-alkynyl, and not the CH₂-group directly attached to the core of the compound of formula (1), can be replaced by O or S, and
C₅₋₈-cycloalkyl, C₅₋₈-cycloalkenyl, 5 to 14 membered heterocycloalkyl and 5 to 14 membered heterocycloalkenyl can be substituted with one to five substituents independently selected from the group consisting of C₁₋₂₀-alkyl, C₂₋₂₀-alkenyl, C₂₋₂₀-alkynyl, C₆₋₁₀-aryl, 5 to 10 membered heteroaryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂,
C₆₋₁₄-aryl can be substituted with one to five substituents independently selected from the group consisting of C₁₋₂₀-alkyl, C₂₋₂₀-alkenyl, C₂₋₂₀-alkynyl, C₅₋₆-cycloalkyl, C₅₋₆-cycloalkenyl, 5 to 10 membered heterocycloalkyl, 5 to 10 membered heterocycloalkenyl, and 5 to 10 membered heteroaryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂,
5 to 14 membered heteroaryl can be substituted with one to five substituents independently selected from the group consisting of C₁₋₂₀-alkyl, C₂₋₂₀-alkenyl, C₂₋₂₀-alkynyl, C₅₋₆-cycloalkyl, C₅₋₆-cycloalkenyl, 5 to 10 membered heterocycloalkyl, 5 to 10 membered heterocycloalkenyl, C₆₋₁₀-aryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂,
   wherein
   R^{a} and R^{b} are independently selected from the group consisting of H, C₁₋₂₀-alkyl, C₂₋₂₀-alkenyl and C₂₋₂₀-alkynyl,
   C₁₋₂₀-alkyl, C₂₋₂₀-alkenyl and C₂₋₂₀-alkynyl can be substituted with one to five substituents selected from the group consisting of phenyl, OR^{c}, OC(O)—R^{c}, C(O)—OR^{c}, C(O)—R^{c}, NR^{c}R^{d}, NR^{c}[C(O)R^{d}], N[C(O)R^{c}][C(O)R^{d}], halogen, CN and NO₂, and,
   C₅₋₆-cycloalkyl, C₅₋₆-cycloalkenyl, 5 to 10 membered heterocycloalkyl, 5 to 10 membered heterocycloalkenyl, C₆₋₁₀-aryl and 5 to 10 membered heteroaryl can be substituted with one to five substituents independently selected from the group consisting of C₁₋₁₀-alkyl, C₂₋₁₀-alkenyl, C₂₋₁₀-alkynyl, OR^{c}, OC(O)—R^{c}, C(O)—OR^{c}, C(O)—R^{c}, NR^{c}R^{d}, NR^{c}[C(O)R^{d}], N[C(O)R^{c}][C(O)R^{d}], halogen, CN and NO₂,
      wherein
      R^{c} and R^{d} are independently selected from the group consisting of H, C₁₋₁₀-alkyl, C₂₋₁₀-alkenyl and C₂₋₁₀-alkynyl,
         wherein
         C₁₋₁₀-alkyl, C₂₋₁₀-alkenyl and C₂₋₁₀-alkynyl can be substituted with one to five substituents selected from the group consisting of halogen, CN and NO₂.

Also part of the present invention is an electronic device comprising the compound of formula 1. Preferably, the electronic device is an organic field effect transistor (OFET).

Usually, an organic field effect transistor comprises a dielectric layer, a semiconducting layer and a substrate. In addition, an organic field effect transistor usually comprises a gate electrode and source/drain electrodes.

Preferably, the semiconducting layer comprises the compound of formula 1. The semiconducting layer can have a thickness of 5 to 500 nm, preferably of 10 to 100 nm, more preferably of 20 to 50 nm.

The dielectric layer comprises a dielectric material. The dielectric material can be silicon dioxide or aluminium oxide, or, an organic polymer such as polystyrene (PS), poly(methylmethacrylate) (PMMA), poly(4-vinylphenol) (PVP), poly(vinyl alcohol) (PVA), benzocyclobutene (BCB), or polyimide (PI). The dielectric layer can have a thickness of 10 to 2000 nm, preferably of 50 to 1000 nm, more preferably of 100 to 800 nm.

The dielectric layer can in addition to the dielectric material comprise a self-assembled monolayer of organic silane derivates or organic phosphoric acid derivatives. An example of an organic silane derivative is octyltrichlorosilane. An examples of an organic phosphoric acid derivative is octyldecylphosphoric acid. The self-assembled monolayer comprised in the dielectric layer is usually in contact with the semiconducting layer.

The source/drain electrodes can be made from any suitable source/drain material, for example gold (Au) or tantalum (Ta). The source/drain electrodes can have a thickness of 1 to 100 nm, preferably from 20 to 70 nm.

The gate electrode can be made from any suitable gate material such as highly doped silicon, aluminium (Al), tungsten (W), indium tin oxide, gold (Au) and/or tantalum (Ta). The gate electrode can have a thickness of 1 to 200 nm, preferably from 5 to 100 nm.

The substrate can be any suitable substrate such as glass, or a plastic substrate such as polyethersulfone, polycarbonate, polysulfone, polyethylene terephthalate (PET) and polyethylene naphthalate (PEN). Depending on the design of the organic field effect transistor, the gate electrode, for example highly doped silicon can also function as substrate.

The organic field effect transistor can be prepared by methods known in the art.

For example, a bottom-gate organic field effect transistor can be prepared as follows: The dielectric material, for example Al₂O₃ or silicon dioxide, can be applied as a layer on a gate electrode such as highly doped silicon wafer, which also functions as substrate, by a suitable deposition method such as atom layer deposition or thermal evaporation. A self-assembled monolayer of an organic phosphoric acid derivative or an organic silane derivative can be applied to the layer of the dielectric material. For example, the organic phosphoric acid derivative or the organic silane derivative can be applied from solution using solution-deposition techniques. The semiconducting layer can be formed by either solution deposition or thermal evaporation in vacuo of a compound of formula 1 on the self-assembled monolayer of the organic phosphoric acid derivative or the organic silane derivative. Source/drain electrodes can be formed by deposition of a suitable source/drain material, for example tantalum (Ta) and/or gold (Au), on the semiconducting layer through a shadow masks. The channel width (W) is typically 50 μm and the channel length (L) is typically 1000 μm.

Also part of the invention is the use of the compound of formula 1 as semiconducting material.

The compounds of formula 1 show a high chemical stability under ambient temperature, air and light conditions. A high chemical stability means that no or almost no chemical modifications, such as oxidation, degradation or dimerization, of the compounds of formula 1 is observed over time. In addition, the compounds of formula 1 are stable upon heating to temperatures up to 70° C. for up to several hours, for example the time required to measure a ¹³C NMR, without noticable decomposition.

In addition, the compounds of formula 1 are soluble in organic solvents and thus are compatible with liquid processing techniques. For example, the compound of formula 1b is soluble in common organic solvents such as chloroform, toluene and tetrahydrofurane at ambient temperature. The compounds of formulae 1c and 1d dissolve well in warm organic solvents.

Furthermore, the compounds of formula 1 are suitable as semiconducting material in electronic devices, in particular in organic field effect transistors (OFETs).

### EXAMPLES

### Example 1

### Preparation of Compound 1a

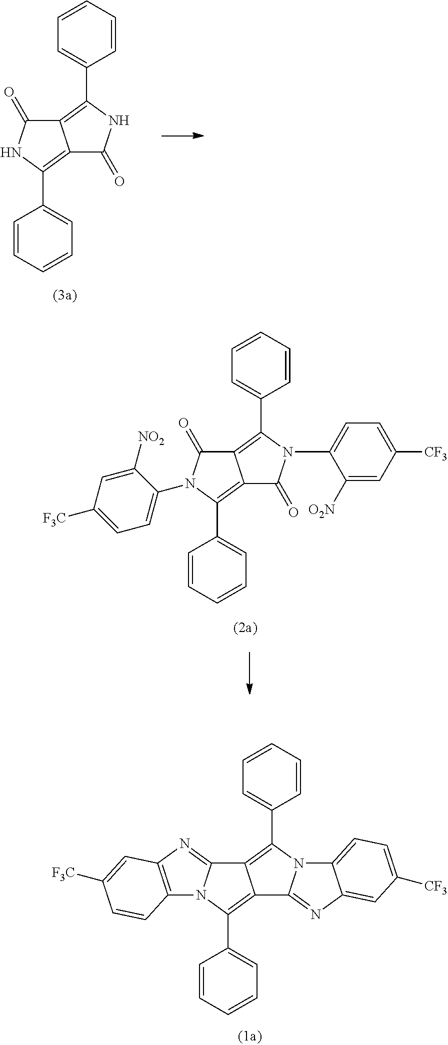 [SMILES: O=C1NC(c2ccccc2)=C2C(=O)NC(c3ccccc3)=C12 ; O=C1C2=C(c3ccccc3)N(c3ccc(C(F)(F)F)cc3[N+](=O)[O-])C(=O)C2=C(c2ccccc2)N1c1ccc(C(F)(F)F)cc1[N+](=O)[O-] ; FC(F)(F)c1ccc2c(c1)nc1c3c(-c4ccccc4)n4c5ccc(C(F)(F)F)cc5nc4c3c(-c3ccccc3)n21]

### Preparation of Compound 3a

Compound 3a was prepared as described by Potrawa, T.; Langhals, H. Chem. Ber. 1987, 120, 1075-1078.

### Preparation of Compound 2a

A mixture of DPP 3a (400 mg, 1.39 mmol), 2-fluoro-5-trifluoromethyl-nitrobenzene (0.78 mL, 5.56 mmol) and K₂CO₃ (770 mg, 5.56 mmol) in DMF (200 mL) was stirred at 70° C. for 22 h, and the suspension became clear when the reaction is finished. Afterwards K₂CO₃ was removed by filtration and the solvent was removed by reduced pressure to get the crude product. Methanol (10 mL) was added to the crude product and the precipitate was filtered, washed with methanol (10 mL) until it became colorless, and dried under vacuum to get compound 2a as a yellow solid (490 mg, 53%).

¹H NMR ([D₆]DMSO, 400 MHz, 300 K): δ=8.60 (d, ⁴J=1.8 Hz, 0.9H), 8.53 (d, ⁴J=1.8 Hz, 1.1H), 8.25 (dd, ⁴J=1.8 Hz, ³J=8.7 Hz, 1.1H), 8.18 (dd, ⁴J=1.6 Hz, ³J=8.4 Hz, 0.9H), 7.86 (d, ³J=8.1 Hz, 1.1H), 7.62-7.44 (m, 10.9H); the ratio of the isomers is ≈0.9/1.1. ¹³C NMR ([D₆]DMSO, 150 MHz, 343 K): δ=161.9, 159.4, 159.3, 146.5, 145.9, 145.7, 132.7, 132.0, 131.9, 131.8, 131.5, 131.4, 130.6, 130.54, 130.52, 129.8, 129.5, 128.9, 128.75, 128.67, 128.6, 125.7, 125.6, 125.0, 123.2, 122.8, 122.7, 122.54, 122.51, 121.4, 109.84, 109.79. MS (MALDI TOF, neg. mode, CHCl₃): m/z: calculated for C₃₂H₁₆F₆N₄O₆: 666.105 [M]⁻, found: 666.039. HRMS (ESI, pos. mode, acetonitrile/chloroform 1:1): m/z: calculated for C₃₂H₁₇F₆N₄O₆: 667.1052 [M+H]⁺, found: 667.1050. CV (CH₂Cl₂, 0.1 M TBAHFP, vs. Fc/Fc⁺): E^{red} (X/X⁻)=−1.43 V, E_{1/2}^{ox} (X/X⁺)=1.04 V. UV-Vis (CHCl₃): λₘₐₓ/nm (ε)=464 (23800 M⁻¹ cm⁻¹).

### Preparation of Compound 1a

A mixture of compound 2a (120 mg, 0.18 mmol), and SnCl₂.2H₂O (410 mg, 1.8 mmol) in ethyl acetate (25 mL) was heated at 78° C. for 3 h under argon. After the solution was cooled down to room temperature, the pH is made slightly basic (pH 7-8) by addition of 10% aqueous sodium bicarbonate before extracting with ethyl acetate. The organic phase was collected and dried over magnesium sulfate, and the solvent was removed under reduced pressure to get the crude product, which was washed with methanol (5 mL) and dried under vacuum to get the desired intermediate product diamine-DPP, which was used for the next step without further purification. The crude diamine-DPP from previous step (110 mg) and DABCO (90 mg, 0.80 mmol) were dissolved in mesitylene (70 mL) while heating to 120° C. for 10 min. Titanium tetrachloride (0.14 mL, 1.30 mmol) in mesitylene (2 mL) was added dropwise to the reaction mixture and the solution was kept at 120° C. for additional 30 min. The hot reaction was dropped quickly in to 50 mL water, extracted with a small amount of ethyl acetate, and the organic phase was passed through a neutral aluminum oxide column using chloroform as eluent. The solvent was removed under vacuum and the residue was washed with 2 mL methanol to obtain the pure compound 1a as a red solid (15 mg). The total yield of the two steps from compound 2a to 1a is 15%.

¹H NMR ([D₂]tetrachloroethane, 600 Hz, 353 K): δ=8.15 (d, ³J=7.4 Hz, 4H), 7.96 (s, 2H), 7.70-7.65 (m, 6H), 7.45 (d, 3J=8.5 Hz, 2H), 7.39 (d, 3J=8.5 Hz, 2H). ¹³C NMR ([D₂]tetrachloroethane, 150 Hz, 343 K): δ=139.3, 133.5, 132.4, 129.6, 129.5, 127.2, 126.0, 125.6, 124.0, 121.4, 121.3, 120.6, 119.1, 119.1, 120.0, 117.7, 116.9, 116.7, 116.5, 112.2. MS (MALDI TOF, neg. mode, CHCl₃): m/z: calculated for C₃₂H₁₆F₆N₄: 570.128 [M]⁻, found: 570.110. HRMS (ESI, pos. mode, acetonitrile/chloroform 1:1): m/z: calculated for C₃₂H₁₇F₆N₄: 571.1357 [M+H]⁺, found: 571.1353. CV (CH₂Cl₂, 0.1 M TBAHFP, vs. Fc/Fc⁺): E_{1/2}^{red} (X/X⁻)=−1.31 V, E^{ox} (X/X⁺)=0.99 V. UV-Vis (CHCl₃): λₘₐₓ/nm (ε)=484 (15700 M⁻¹ cm⁻¹).

### Example 2

### Preparation of Compound 1b

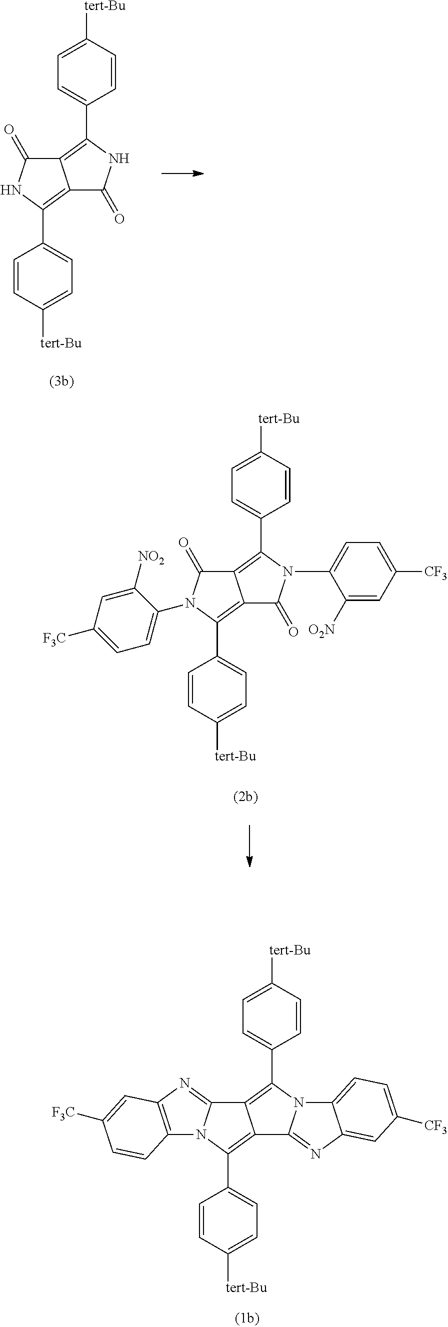

### Preparation of Compound 3b

Compound 3b was prepared as described by Potrawa, T.; Langhals, H. Chem. Ber. 1987, 120, 1075-1078.

### Preparation of Compound 2b

A mixture of DPP 3b (200 mg, 0.5 mmol), 2-fluoro-5-trifluoromethyl-nitrobenzene (0.28 mL, 2 mmol) and K₂CO₃ (276 mg, 2 mmol) in DM F (100 mL) was stirred at 70° C. for 24 h. After the reaction solution was cooled down to room temperature, K₂CO₃ was removed by filtration and the solvent was removed under reduced pressure to get the crude product. Methanol was added to the latter and the precipitate was filtered, washed with methanol until it became colorless and dried under vacuum to obtain compound 2b as a red solid (195 mg, 50%).

¹H NMR: (CDCl₃, 400 Hz, 300 K): δ=8.39 (d, ⁴J=1.7 Hz, 0.8H), 8.34 (d, ⁴J=1.8 Hz, 1.2H), 7.91 (dd, ⁴J=1.6 Hz, ³J=8.4 Hz, 1.2H), 7.79 (dd, ⁴J=1.6 Hz, ³J=8.4 Hz, 0.8H), 7.60-7.58 (m, 1.6H), 7.54-7.48 (m, 3.6H), 7.40-7.36 (m, 4H), 7.20 (d, ³J=8.3 Hz, 0.8H), 1.30 (s, 7.2H), 1.29 (s, 10.8H). The ratio of the isomers is ≈0.8/1.2. ¹³C NMR (CDCl₃, 100 Hz, 300 K): δ=160.3, 160.1, 156.4, 156.3, 146.8, 146.7, 146.3, 145.9, 132.7, 132.6, 131.9, 131.5, 131.2, 131.1, 130.9, 130.3, 130.26, 130.23, 129.8, 129.5, 129.2, 126.14, 126.09 123.8, 123.4, 123.2, 122.99, 122.95 121.1, 110.8, 110.5, 31.0, 30.9. HRMS (ESI, pos. mode, acetonitrile/chloroform 1:1): m/z: calculated for C₄₀H₃₃F₆N₄O₆: 779.2304 [M+H]⁺, found: 779.2301. CV (CH₂Cl₂, 0.1 M TBAHFP, vs. Fc/Fc⁺): E^{red} (X/X⁻)=−1.53 V, E_{1/2}^{ox} (X/X⁺)=0.94 V. UV-Vis (CHCl₃): λₘₐₓ/nm (ε)=483 (29600 M⁻¹ cm⁻¹).

### Preparation of Compound 1b

A mixture of compound 2b (110 mg, 0.14 mmol), and SnCl₂.2H₂O (320 mg, 2.6 mmol) in ethyl acetate (25 mL) was heated at 78° C. for 3 h under argon. After the solution was cooled down to room temperature, the pH is made slightly basic (pH 7-8) by the addition of 10% aqueous sodium bicarbonate before extracting with ethyl acetate. The organic phase was separated and dried over magnesium sulfate, and the solvent was evaporated under reduced pressure to get the crude product, which was washed with methanol and dried under vacuum to get the desired product diamine-DPP, which was used in the next step without further purification. The crude diamine-DPP from the previous step (100 mg) and DABCO (110 mg, 1.0 mmol) were dissolved in mesitylene (100 mL) while heating to 120° C. for 10 min. Titanium tetrachloride (0.18 mL, 1.65 mmol) in mesitylene (2 mL) was added dropwise to the reaction mixture, the solution was kept at 120° C. for about 50 min. After the reaction was finished, the hot solution was dropped immediately into 50 mL water, and extracted with a small amount of ethyl acetate. The organic phase was passed through a neutral aluminum oxide column using chloroform as eluent and the solvent was removed under reduced pressure. The residue was washed with methanol (2 mL) to get the compound 1b as a red solid (13 mg). The total yield of the two steps from 2b to 1b is 14%.

¹H NMR (CDCl₃, 400 Hz, 300 K): δ=8.12 (d, ³J=8.3 Hz, 4H), 8.00 (s, 2H), 7.75 (d, ³J=8.5 Hz, 2H), 7.56 (d, ³J=8.5 Hz, 2H). 7.43 (d, ³J=8.5 Hz, 2H), 1.26 (s, 18H). ¹³C NMR (CDCl₃, 100 Hz, 300 K): δ=155.1, 152.4, 148.3, 138.1, 135.4, 132.1, 128.0, 125.5, 125.0, 124.7, 124.6, 123.0, 122.0, 119.9, 117.6, 117.6, 115.7, 111.1, 30.2, 28.7. MS (MALDI TOF, neg. mode, CHCl₃): m/z: calculated for C₄₀H₃₂F₆N₄: 682.253 [M]⁻, found: 682.232. HRMS (ESI, pos. mode, acetonitrile/chloroform 1:1): m/z: calculated for C₄₀H₃₃F₆N₄: 683.2610 [M+H]⁺, found: 683.2610. CV (CH₂Cl₂, 0.1 M TBAHFP, vs. Fc/Fc⁺): E_{1/2}^{red} (X/X⁻)=−1.39 V, E^{ox} (X/X⁺)=0.96 V. UV-Vis (CHCl₃): λₘₐₓ/nm (ε)=494 (24900 M⁻¹ cm⁻¹).

### Example 3

### Preparation of Compound 1c

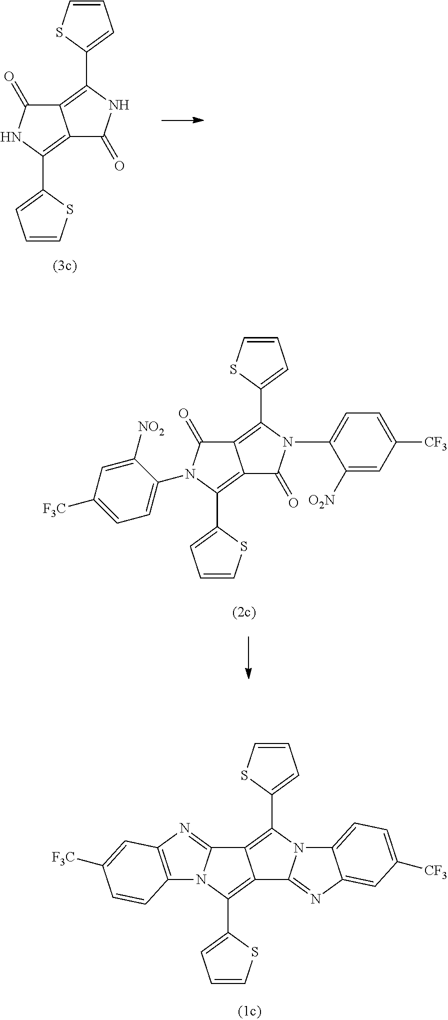 [SMILES: O=C1NC(c2cccs2)=C2C(=O)NC(c3cccs3)=C12 ; O=C1C2=C(c3cccs3)N(c3ccc(C(F)(F)F)cc3[N+](=O)[O-])C(=O)C2=C(c2cccs2)N1c1ccc(C(F)(F)F)cc1[N+](=O)[O-] ; FC(F)(F)c1ccc2c(c1)nc1c3c(-c4cccs4)n4c5ccc(C(F)(F)F)cc5nc4c3c(-c3cccs3)n21]

### Preparation of Compound 3c

Compound 3c was prepared as described by Woo, C. H.; Beaujuge, P. M.; Holcombe, T. W.; Lee, O. P.; Fréchet, J. M. J. J. Am. Chem. Soc. 2010, 132, 15547-15549.

### Preparation of Compound 2c

A mixture of DPP 3c (400 mg, 1.33 mmol), 2-fluoro-5-trifluoromethyl-nitrobenzene (0.75 mL, 5.32 mmol) and K₂CO₃ (736 mg, 5.32 mmol) in DMF (50 mL) was stirred at 70° C. for 24 h. After the reaction was cooled down to room temperature, K₂CO₃ was removed by filtration, extracted twice with 5 mL chloroform, and the collected solvent was removed under reduced pressure. Methanol was added to the crude product and the precipitate was filtered, and washed with methanol until it became colorless, and dried under vacuum to get compound 2c as a red solid (489 mg, 54%).

¹H NMR ([D₆]DMSO, 600 Hz, 350 K): δ=8.69 (d, ⁴J=2.0 Hz, 0.8H), 8.66 (d, ⁴J=2.0 Hz, 1.2H), 8.38-8.36 (m, 4H), 8.25 (d, ³J=8.2 Hz, 1.2H), 8.10 (d, ³J=8.2 Hz, 0.8H), 7.95-7.93 (m, 2H), 7.29-7.27 (m, 2H); the ratio of the isomers is ≈0.8/1.2. ¹³C NMR ([D₆]DMSO, 150 Hz, 343 K): δ=161.9, 159.2, 159.1, 147.3, 147.2, 138.7, 38.6, 134.8, 134.7, 134.6, 134.30, 134.25, 134.1, 131.5, 131.3, 131.2, 131.12, 131.08, 128.4, 128.3, 128.2, 123.2, 123.0, 122.9, 122.8, 122.7, 121.4, 107.2. MS (MALDI TOF, neg. mode, CHCl₃): m/z: calculated for C₂₈H₁₂F₆N₄O₆S₂: 678.010 [M]⁻, found: 678.991. HRMS (ESI, pos. mode, acetonitrile/chloroform 1:1): m/z calculated for C₂₈H₁₃F₆N₄O₆S₂: 679.0181 [M+H]⁺, found: 679.0174. CV (CH₂Cl₂, 0.1 M TBAHFP, vs. Fc/Fc⁺): E^{red} (X/X⁻)=□−1.37 V, E_{1/2}^{ox} (X/X⁺)=0.76 V, E_{1/2}^{ox} (X⁺/X²⁺)=1.09 V. UV-Vis (CHCl₃): λₘₐₓ/nm (ε)=502 (29100), 537 (33000 M⁻¹ cm⁻¹).

### Preparation of Compound 1c

A mixture of compound 2c (130 mg, 0.20 mmol), and SnCl₂.2H₂O (344 mg, 2.6 mmol) in ethyl acetate (30 mL) was heated at 70° C. for 3 h under argon. After the solution was cooled down to room temperature, the pH was made slightly basic (pH 7-8) by the addition of 10% aqueous sodium bicarbonate before extracting with ethyl acetate. The organic phase was separated and dried over magnesium sulfate. The solvent was removed under reduced pressure and the residue was dried under vacuum to get the desired product diamine-DPP, which was used for the next step without further purification.

The crude product from the previous step (120 mg) and DABCO (140 mg, 1.25 mmol) were dissolved in mesitylene (150 mL) while heating to 125° C. for 10 min. Titanium tetrachloride (0.18 mL, 1.63 mmol) in mesitylene (2 mL) was added dropwise to the reaction mixture, the solution was kept at 125° C. for about 60 min. The hot reaction solution was dropped quickly into 50 mL water, extracted with a small amount of ethyl acetate, and the organic phase was passed through a neutral aluminum oxide column using chloroform as eluent. The solvent was removed under reduced pressure and the residue was washed with methanol to get pure 1c as a dark brown solid (22 mg). The total yield of the two steps from 2c to 1c is 20%.

¹H NMR ([D₂]tetrachloroethane, 600 Hz, 350 K): δ=8.50 (dd, ³J=3.7 Hz, ⁴J=0.8 Hz, 2H), 8.01-8.0 (m, 4H), 7.78 (dd, 3J=5.0 Hz, 4J=0.9 Hz, 2H), 7.46 (dd, 3J=8.5 Hz, 4J=1.0 Hz, 2H), 7.42-7.40 (m, 2H). ¹³C NMR ([D₂]tetrachloroethane, 150 Hz, 343 K): δ=153.3, 149.7, 134.8, 133.2, 132.3, 131.9, 129.6, 128.9, 126.4, 126.1, 125.6, 123.9, 123.8, 121.30, 121.28, 120.6, 119.12, 119.09, 116.7, 116.5, 116.2, 112.5, 99.9, 80.1, 80.0, 79.8. MS (MALDI TOF, neg. mode, CHCl₃): m/z: calculated for C₂₈H₁₂F₆N₄S₂: 582.041 [M]^{-□}, found: 582.002. HRMS (ESI, pos. mode, acetonitrile/chloroform 1:1): m/z: calculated for C₂₈H₁₃F₆N₄S₂: 583.0486. [M+H]⁺, found: 583.0483. CV (CH₂Cl₂, 0.1 M TBAHFP, vs. Fc/Fc⁺): E_{1/2}^{red} (X/X⁻)=−1.27 V, E^{ox} (X/X⁺)=0.84 V, E^{ox} (X⁺/X²⁺)=1.02 V. UV-Vis (CHCl₃): λₘₐₓ/nm (ε)=527 (25500 M⁻¹ cm⁻¹).

### Example 4

### Preparation of Compound 1d

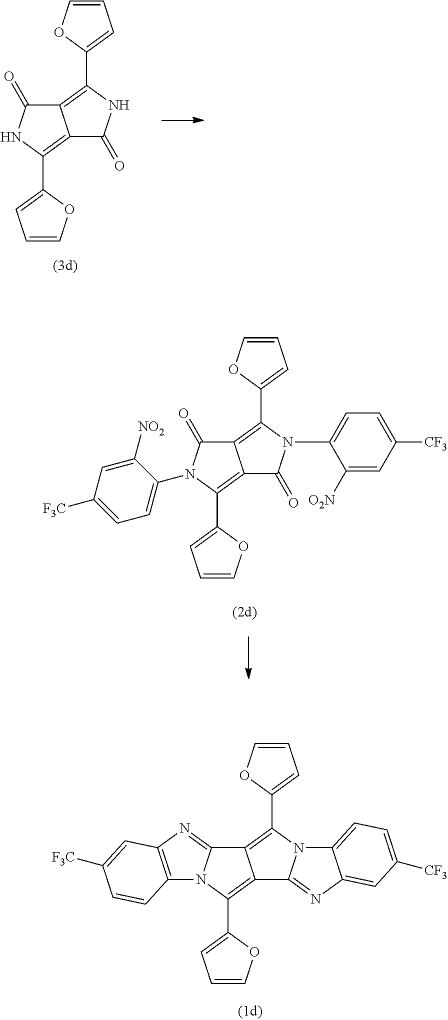 [SMILES: O=C1NC(c2ccco2)=C2C(=O)NC(c3ccco3)=C12 ; O=C1C2=C(c3ccco3)N(c3ccc(C(F)(F)F)cc3[N+](=O)[O-])C(=O)C2=C(c2ccco2)N1c1ccc(C(F)(F)F)cc1[N+](=O)[O-] ; FC(F)(F)c1ccc2c(c1)nc1c3c(-c4ccco4)n4c5ccc(C(F)(F)F)cc5nc4c3c(-c3ccco3)n21]

### Preparation of Compound 3d

Compound 3d was prepared as described by Woo, C. H.; Beaujuge, P. M.; Holcombe, T. W.; Lee, O. P.; Fréchet, J. M. J. J. Am. Chem. Soc. 2010, 132, 15547-15549.

### Preparation of Compound 2d

A mixture of DPP 3d (400 mg, 1.5 mmol), 2-fluoro-5-trifluoromethyl-nitrobenzene (0.64 mL, 6.0 mmol) and K₂CO₃ (828 mg, 3.0 mmol) in DMF (160 mL) was stirred at 70° C. for 5 h. K₂CO₃ was removed by filtration and the solvent was removed under reduced pressure. Methanol was added to the crude product and the solid was filtered, washed with methanol until it became colorless and dried under vacuum, affording 2d as a red solid (550 mg, 57%).

¹H NMR ([D₆]DMSO, 600 Hz, 300 K): δ=8.66 (d, ⁴J=2.0 Hz, 0.8H), 8.63 (d, ⁴J=2.0 Hz, 1.2H), 8.39-8.36 (m, 2H), 8.22 (d, ³J=8.2 Hz, 1.2H), 8.08 (d, ³J=8.2 Hz, 0.8H), 7.89 (dd, ³J=1.6 Hz, 4J=0.5 Hz, 0.8H), 7.87 (dd, 3J=1.6 Hz, 4J=0.5 Hz, 1.2H), 7.80-7.79 (m, 2H), 6.84-6.82 (m, 2H); the ratio of the isomers ≈0.8/1.2. ¹³C NMR ([D₆]DMSO, 150 Hz, 343 K): δ=158.9, 148.6, 148.4, 146.7, 146.6, 142.5, 142.3, 133.7, 133.4, 132.7, 132.6, 132.3, 131.2, 131.1, 130.6, 130.4, 123.7, 123.0, 122.8, 122.7, 121.9, 121.8, 120.6, 120.4, 114.1, 114.0, 105.8, 54.9. HRMS (ESI, pos. mode, acetonitrile/chloroform 1:1): m/z calculated for C₂₈H₁₃F₆N₄O₈: 647.0638, [M+H]⁺, found: 647.0603. CV (CH₂Cl₂, 0.1 M TBAHFP, vs. Fc/Fc⁺): E^{red} (X/X⁻)=−1.39 V, E_{1/2}^{ox} (X/X⁺)=0.75 V, E_{1/2}^{ox} (X⁺/X²⁺)=0.94 V. UV-Vis (CHCl₃): λₘₐₓ/nm (ε)=492 (31400), 530 (46700 M⁻¹ cm⁻¹).

### Preparation of Compound 1d

This compound was synthesized from compound 2d by using the same procedure as applied for the synthesis of compound 1c from 2c. Yield of 1d: 17%.

¹H NMR ([D₂]tetrachloroethane, 600 Hz, 350 K): δ=8.39 (d, ³J=2.9 Hz, 2H), 8.28 (d, ³J=8.4 Hz, 2H), 7.98 (s, 2H), 7.86 (2H), 7.52 (d, ³J=8.4 Hz, 2H). 6.89-6.88 (m, 2H). MS (MALDI TOF, neg. mode, CHCl₃): m/z: calculated for C₂₈H₁₂F₆N₄O₂: 550.086 [M]⁻, found: 550.013. HRMS (ESI, pos. mode, acetonitrile/chloroform 1:1): m/z: calculated for C₂₈H₁₃F₆N₄O₂: 551.0943 [M+H]⁺, found: 551.0936. CV (CH₂Cl₂, 0.1 M TBAHFP, vs. Fc/Fc⁺): E_{1/2}^{red} (X/X⁻)=−1.27 V, E^{ox} (X/X⁺)=0.72 V, E^{ox} (X⁺/X²⁺)=1.06 V. UV-Vis (CHCl₃): λₘₐₓ/nm (ε)=570 (28500 M⁻¹ cm⁻¹).

### Example 5

### Test of the Chemical Stability of Compound 1a

Compound 1a was dissolved in chloroform (c=9.0×10⁻⁶ M), and stored under ambient air and light conditions at room temperature. UV-Vis absorption spectra of compound 1a, dissolved in chloroform (c=9.0×10⁻⁶ M), were recorded after 0, 1, 2, 3 and 4 days of storage at room temperature in a conventional quartz cell (light pass 10 mm) on a Perkin-Elmer Lambda 950 spectrometer. No chemical modification, such as degradation, was observed after 4 days.

### Example 6

### Preparation of Transistors Comprising Compound 1c as Semiconducting Material

Highly doped p-type silicon (100) wafers (0.01-0.02 Ω·cm) were used as substrates A. Highly doped p-type silicon (100) wafers (0.005-0.02 Ω·cm) with a 100 nm thick thermally grown SiO₂ layer (capacitance 34 nF/cm²) were used as substrates B.

Onto substrates A, a 30 nm thick layer of aluminum is deposited by thermal evaporation in a Leybold UNIVEX 300 vacuum evaporator from a tungsten wire, at a pressure of 2×10⁻⁶ mbar and with an evaporation rate of 1 nm/s. The surface of the aluminum layer is oxidized by a brief exposure to an oxygen plasma in an Oxford reactive ion etcher (RIE, oxygen flow rate: 30 sccm, pressure: 10 mTorr, plasma power: 200 W, plasma duration 30 sec) and the substrate is then immersed into a 2-propanol solution of a phosphonic acid (1 mMol solution of C₁₄H₂₉PO(OH)₂ [TDPA] or 1 mMol solution of C₇F₁₅C₁₁H₂₂PO(OH)₂ [FODPA]) and left in the solution for 1 hour, which results in the formation of a self-assembled monolayer (SAM) of phosphonic acid molecules on the aluminum oxide surface. The substrate is taken out of the solution and rinsed with pure 2-propanol, dried in a stream of nitrogen and left for 10 min on a hotplate at a temperature of 100° C. The total capacitance of the AlOₓ/SAM gate dielectric on substrate A is 810 nF/cm² in case of C₁₄H₂₉PO(OH)₂ and 710 nF/cm² in case of C₇F₁₅C₁₁H₂₂PO(OH)₂.

On substrates B, an about 8 nm thick layer of Al₂O₃ is deposited by atomic layer deposition in a Cambridge NanoTech Savannah (80 cycles at a substrate temperature of 250° C.). The surface of the aluminum oxide layer is activated by a brief exposure to an oxygen plasma in an Oxford reactive ion etcher (RIE, oxygen flow rate: 30 sccm, pressure: 10 mTorr, plasma power: 200 W, plasma duration 30 sec) and the substrate is then immersed into a 2-propanol solution of a phosphonic acid (1 mMol solution of C₁₄H₂₉PO(OH)₂ [TDPA] or 1 mMol solution of C₇F₁₅C₁₁H₂₂PO(OH)₂ [FODPA]) and left in the solution for 1 hour, which results in the formation of a self-assembled monolayer (SAM) of phosphonic acid molecules on the aluminum oxide surface. The substrate is taken out of the solution and rinsed with pure 2-propanol, dried in a stream of nitrogen and left for 10 min on a hotplate at a temperature of 100° C. The total capacitance of the SiO₂/AlOₓ/SAM gate dielectric on substrate B is 32 nF/cm² (independent on the choice of the phosphonic acid).

The contact angle of water on the TDPA-treated substrates is 108°, and on the FODPA-treated substrates 118°.

A 30 nm thick film of the compound 1c is deposited by thermal sublimation in a Leybold UNIVEX 300 vacuum evaporator from a molybdenum boat, at a pressure of 2×10⁻⁶ mbar and with an evaporation rate of 0.3 nm/s.

For the source and drain contacts 30 nm of gold is evaporated through a shadow mask in a Leybold UNIVEX 300 vacuum evaporator from tungsten boat, at a pressure of 2×10⁻⁶ mbar and with an evaporation rate of 0.3 nm/s. The transistors have a channel length (L) ranging from 10 to 100 μm and a channel width (W) ranging from 50 to 1000 μm.

To be able to contact the back side of the silicon wafer, the wafer (which also serves as the gate electrode of the transistors) is scratched on the back side and coated with silver ink.

### Example 7

### Measuring the Electrical Characteristics of the Transistors of Example 6

The electrical characteristics of the transistors of example 6 are measured on a Micromanipulator 6200 probe station using an Agilent 4156C semiconductor parameter analyzer. All measurements are performed in air at room temperature. The probe needles are brought into contact with the source and drain contacts of the transistors by putting them down carefully on top of the gold contacts. The gate electrode is contacted through the metal substrate holder onto which the wafer is placed during the measurements.

To obtain the transfer curve the drain-source voltage (V_{DS}) is held to 3 V (in case of substrate A) or 40 V (in case of substrate B). The gate-source voltage V_{GS} is swept at medium speed from 0 to 3 V in steps of 0.03 V (substrate A) or from 0 to 40 V in steps of 0.4 V (substrate B) and back. The charge-carrier mobility is extracted in the saturation regime from the slope of (I_{D})^{1/2} versus V_{GS}.

To obtain the output characteristics the drain-source voltage (V_{DS}) is swept at medium speed from 0 to 3 V in steps of 0.03 V (substrate A) and from 0 to 40 V in steps of 0.4 V (substrate B), while the gate-source voltage V_{GS} is held at up to 8 different voltages (e.g. 0, 0.5, 1, 1.5, 2, 2.5, 3 V in case of substrate A or 0, 10, 20, 30, 40 V in case of substrate B).

The results are depicted in Table 1.

TABLE 1

| | | | | | Electron | |
|---|---|---|---|---|---|---|
| | | | Substrate | Hole | Mobility | On/Off |
| Organic | | | Temperature | Mobility | μₑ | Ratio |
| Semiconductor | Substrate | SAM | T_{sub} [° C.] | μₚ [cm²/Vs] | [cm²/Vs] | Iₒₙ/I_{off} |
| 1c | B | FODPA | 50 | 10⁻³ | | 10² |
| 1c | B | TDPA | 50 | | 10⁻⁵ | 10 |
| 1c | A | FODPA | 50 | 10⁻³ | | 10² |

1.-10. (canceled)

11. A compound of formula 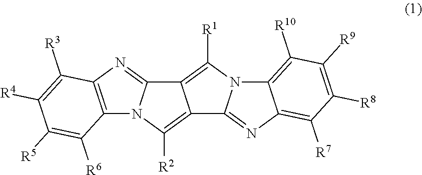
wherein
R¹ and R² are independently from each other selected from the group consisting of H, C₁₋₃₀-alkyl, C₂₋₃₀-alkenyl, C₂₋₃₀-alkynyl, C₅₋₈-cycloalkyl, C₅₋₈-cycloalkenyl, 5 to 14 membered heterocycloalkyl, 5 to 14 membered heterocycloalkenyl, C₆₋₁₄-aryl and 5 to 14 membered heteroaryl, and
R², R³, R⁴, R⁵, R⁶, R⁷, R⁸, R⁹ and R¹⁰ are independently from each other selected from the group consisting of H, C₁₋₃₀-alkyl, C₂₋₃₀-alkenyl, C₂₋₃₀-alkynyl, C₅₋₈-cycloalkyl, C₅₋₈-cycloalkenyl, 5 to 14 membered heterocycloalkyl, 5 to 14 membered heterocycloalkenyl, C₆₋₁₄-aryl, 5 to 14 membered heteroaryl, halogen, CN, —SCN, NO₂, OH, O—C₁₋₃₀-alkyl, O—C₂₋₃₀-alkenyl, O—C₂₋₃₀-alkynyl, O—C₅₋₈-cycloalkyl, O—C₅₋₈-cycloalkenyl, O-5 to 14 membered heterocycloalkyl, O-5 to 14 membered heterocycloalkenyl, O—C₆₋₁₄-aryl, O-5 to 14 membered heteroaryl, SH, S—C₁₋₃₀-alkyl, S—C₂₋₃₀-alkenyl, S—C₂₋₃₀-alkynyl, S—C₅₋₈-cycloalkyl, S—C₅₋₈-cycloalkenyl, S-5 to 14 membered heterocycloalkyl, S-5 to 14 membered heterocycloalkenyl, S—C₆₋₁₄-aryl, S-5 to 14 membered heteroaryl, C(O)H, CO—C₁₋₃₀-alkyl, CO—C₂₋₃₀-alkenyl, CO—C₂₋₃₀-alkynyl, CO—C₅₋₈-cycloalkyl, CO—C₅₋₈-cycloalkenyl, CO-5 to 14 membered heterocycloalkyl, CO-5 to 14 membered heterocycloalkenyl, CO—C₆₋₁₄-aryl, CO-5 to 14 membered heteroaryl, COOH, NH(C₁₋₃₀-alkyl), N(C₁₋₃₀-alkyl)₂, CONH₂, CONH(C₁₋₃₀-alkyl), CON(C₁₋₃₀-alkyl)₂, SO₂OH, SO₂NH₂, SO₂—C₁₋₃₀-alkyl and SO₂—C₆₋₁₄-aryl,
wherein
C₁₋₃₀-alkyl, C₂₋₃₀-alkenyl and C₂₋₃₀-alkynyl, are optionally substituted with one to nine substituents independently selected from the group consisting of C₅₋₆-cycloalkyl, C₅₋₆-cycloalkenyl, 5 to 10 membered heterocycloalkyl, 5 to 10 membered heterocycloalkenyl, C₆₋₁₀-aryl, 5 to 10 membered heteroaryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂; and one or more CH₂-groups, but not adjacent CH₂-groups of C₁₋₃₀-alkyl, C₂₋₃₀-alkenyl and C₂₋₃₀-alkynyl, and not the CH₂-group directly attached to the core of the compound of formula (1), is optionally replaced by O or S, and
C₅₋₈-cycloalkyl, C₅₋₈-cycloalkenyl, 5 to 14 membered heterocycloalkyl and 5 to 14 membered heterocycloalkenyl are optionally substituted with one to five substituents independently selected from the group consisting of C₁₋₂₀-alkyl, C₂₋₂₀-alkenyl, C₂₋₂₀-alkynyl, C₁₋₁₀-aryl, 5 to 10 membered heteroaryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂,
C₆₋₁₄-aryl is optionally substituted with one to five substituents independently selected from the group consisting of C₁₋₂₀-alkyl, C₂₋₂₀-alkenyl, C₂₋₂₀-alkynyl, C₅₋₆-cycloalkyl, C₅₋₆-cycloalkenyl, 5 to 10 membered heterocycloalkyl, 5 to 10 membered heterocycloalkenyl, and 5 to 10 membered heteroaryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂,
5 to 14 membered heteroaryl is optionally substituted with one to five substituents independently selected from the group consisting of C₁₋₂₀-alkyl, C₂₋₂₀-alkenyl, C₂₋₂₀-alkynyl, C₅₋₆-cycloalkyl, C₅₋₆-cycloalkenyl, 5 to 10 membered heterocycloalkyl, 5 to 10 membered heterocycloalkenyl, C₆₋₁₀-aryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂,
wherein
R^{a} and R^{b} are independently selected from the group consisting of H, C₁₋₂₀-alkyl, C₂₋₂₀-alkenyl and C₂₋₂₀-alkynyl,
C₁₋₂₀-alkyl, C₂₋₂₀-alkenyl and C₂₋₂₀-alkynyl are optionally substituted with one to five substituents independently selected from the group consisting of phenyl, OR^{c}, OC(O)—R^{c}, C(O)—OR^{c}, C(O)—R^{c}, NR^{c}R^{d}, NR^{c}[C(O)R^{d}], N[C(O)R^{c}][C(O)R^{d}], halogen, CN and NO₂, and,
C₅₋₆-cycloalkyl, C₅₋₆-cycloalkenyl, 5 to 10 membered heterocycloalkyl, 5 to 10 membered heterocycloalkenyl, C₆₋₁₀-aryl and 5 to 10 membered heteroaryl are optionally substituted with one to five substituents independently selected from the group consisting of C₁₋₁₀-alkyl, C₂₋₁₀-alkenyl, C₂₋₁₀-alkynyl, OR^{c}, OC(O)—R^{c}, C(O)—OR^{c}, C(O)—R^{c}, NR^{c}R^{d}, NR^{c}[C(O)R^{d}], N[C(O)R^{c}][C(O)R^{d}], halogen, CN and NO₂,
wherein
R^{c} and R^{d} are independently selected from the group consisting of H, C₁₋₁₀-alkyl, C₂₋₁₀-alkenyl and C₂₋₁₀-alkynyl,
wherein
C₁₋₁₀-alkyl, C₂₋₁₀-alkenyl and C₂₋₁₀-alkynyl are optionally substituted with one to five substituents independently selected from the group consisting of halogen, CN and NO₂.

12. The compound of claim 11, wherein R¹ and R² are independently from each other selected from the group consisting of H, C₁₋₃₀-alkyl, C₆₋₁₄-aryl, and 5 to 14 membered heteroaryl, and
R², R³, R⁴, R⁵, R⁶, R⁷, R⁸, R⁹ and R¹⁰ are independently from each other selected from the group consisting of H, C₁₋₃₀-alkyl, C₆₋₁₄-aryl, 5 to 14 membered heteroaryl, halogen, CN, —SCN, NO₂, OH, O—C₁₋₃₀-alkyl, O—C₆₋₁₄-aryl, O-5 to 14 membered heteroaryl, SH, S—C₁₋₃₀-alkyl, S—C₆₋₁₄-aryl, S-5 to 14 membered heteroaryl, C(O)H, CO—C₁₋₃₀-alkyl, CO—C₆₋₁₄-aryl, CO-5 to 14 membered heteroaryl, COOH, NH(C₁₋₃₀-alkyl), N(C₁₋₃₀-alkyl)₂, CONH₂, CONH(C₁₋₃₀-alkyl), CON(C₁₋₃₀-alkyl)₂, SO₂OH, SO₂NH₂, SO₂—C₁₋₃₀-alkyl and SO₂—C₆₋₁₄-aryl,
wherein
C₁₋₃₀-alkyl is optionally substituted with one to nine substituents independently selected from the group consisting of C₆₋₁₀-aryl, 5 to 10 membered heteroaryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂; and one or more CH₂-groups, but not adjacent CH₂-groups of C₁₋₃₀-alkyl and not the CH₂-group directly attached to the core of the compound of formula (1), is optionally replaced by O or S, and
C₆₋₁₄-aryl is optionally substituted with one to five substituents independently selected from the group consisting of C₁₋₂₀-alkyl, 5 to 10 membered heteroaryl OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂,
5 to 14 membered heteroaryl is optionally substituted with one to five substituents independently selected from the group consisting of C₁₋₂₀-alkyl, C₆₋₁₀-aryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂,
wherein
R^{a} and R^{b} are independently selected from the group consisting of H and C₁₋₂₀-alkyl,
C₁₋₂₀-alkyl is optionally substituted with one to five substituents selected from the group consisting of phenyl, OR^{c}, OC(O)—R^{c}, C(O)—OR^{c}, C(O)—R^{c}, NR^{c}R^{d}, NR^{c}[C(O)R^{d}], N[C(O)R^{c}][C(O)R^{d}], halogen, CN and NO₂, and,
C₆₋₁₀-aryl and 5 to 10 membered heteroaryl are optionally substituted with one to five substituents independently selected from the group consisting of C₁₋₁₀-alkyl, OR^{c}, OC(O)—R^{c}, C(O)—OR^{c}, C(O)—R^{c}, NR^{c}R^{d}, NR^{c}[C(O)R^{d}], N[C(O)R^{c}][C(O)R^{d}], halogen, CN and NO₂,
wherein
R^{c} and R^{d} are independently selected from the group consisting of H and C₁₋₁₀-alkyl,
wherein
C₁₋₁₀-alkyl is optionally substituted with one to five substituents independently selected from the group consisting of halogen, CN and NO₂.

13. The compound of claim 11, wherein
R¹ and R² are independently from each other selected from the group consisting of C₆₋₁₄-aryl, and 5 to 14 membered heteroaryl, and
R², R³, R⁴, R⁵, R⁶, R⁷, R⁸, R⁹ and R¹⁰ are independently from each other selected from the group consisting of H and C₁₋₃₀-alkyl,
wherein
C₁₋₃₀-alkyl is optionally substituted with one to nine substituents independently selected from the group consisting of C₆₋₁₀-aryl, 5 to 10 membered heteroaryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂; and one or more CH₂-groups, but not adjacent CH₂-groups of C₁₋₃₀-alkyl and not the CH₂-group directly attached to the core of the compound of formula (1), is optionally replaced by O or S, and
C₆₋₁₄-aryl is optionally substituted with one to five substituents independently selected from the group consisting of C₁₋₂₀-alkyl, 5 to 10 membered heteroaryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂,
5 to 14 membered heteroaryl is optionally substituted with one to five substituents independently selected from the group consisting of C₁₋₂₀-alkyl, C₆₋₁₀-aryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂,
wherein
R^{a} and R^{b} are independently selected from the group consisting of H and C₁₋₂₀-alkyl,
C₁₋₂₀-alkyl is optionally substituted with one to five substituents selected from the group consisting of phenyl, OR^{c}, OC(O)—R^{c}, C(O)—OR^{c}, C(O)—R^{c}, NR^{c}R^{d}, NR^{c}[C(O)R^{d}], N[C(O)R^{c}][C(O)R^{d}], halogen, CN and NO₂, and,
C₆₋₁₀-aryl and 5 to 10 membered heteroaryl are optionally substituted with one to five substituents independently selected from the group consisting of C₁₋₁₀-alkyl, OR^{c}, OC(O)—R^{c}, C(O)—OR^{c}, C(O)—R^{c}, NR^{c}R^{d}, NR^{c}[C(O)R^{d}], N[C(O)R^{c}][C(O)R^{d}], halogen, CN and NO₂,
wherein
R^{c} and R^{d} are independently selected from the group consisting of H and C₁₋₁₀-alkyl,
wherein
C₁₋₁₀-alkyl is optionally substituted with one to five substituents independently selected from the group consisting of halogen, CN and NO₂.

14. The compound of claim 11, wherein
R¹ and R² are independently from each other selected from the group consisting of C₆₋₁₄-aryl, and 5 to 14 membered heteroaryl, and
R², R³, R⁴, R⁵, R⁶, R⁷, R⁸, R⁹ and R¹⁰ are independently from each other selected from the group consisting of H and C₁₋₃₀-alkyl,
wherein
C₁₋₃₀-alkyl is optionally substituted with one to nine substituents independently selected from halogen, and
C₆₋₁₄-aryl is optionally substituted with one to five substituents independently selected from C₁₋₂₀-alkyl.

15. The compound of claim 11, wherein
R¹ and R² are independently from each other selected from the group consisting of phenyl, 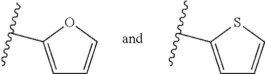 [SMILES: *c1ccco1 ; *c1cccs1]
which are optionally substituted with one or two substituents independently selected from C₁₋₂₀-alkyl,
and
R², R³, R⁴, R⁵, R⁶, R⁷, R⁸, R⁹ and R¹⁰ are independently from each other selected from the group consisting of H and CF₃.

16. A process for the preparation of the compound of claim 11, which process comprises the steps of
i) reducing a compound of formula 2 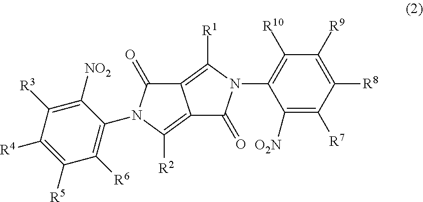
wherein R¹, R², R³, R⁴, R⁵, R⁶, R⁷, R⁸, R⁹ and R¹⁰ are as defined in claim 11,
to the compound of formula 2′ 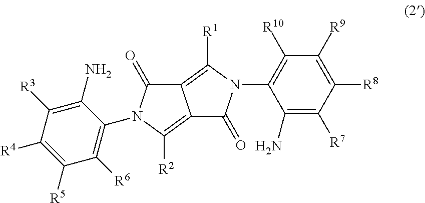
wherein R¹, R², R³, R⁴, R⁵, R⁶, R⁷, R⁸, R⁹ and R¹⁰ are as defined in claim 11,
and
ii) treating the compound of formula 2′ with a suitable catalyst to obtain a compound of formula 1.

17. A compound of formula 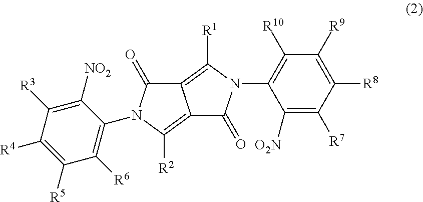
wherein
R¹ and R² are independently from each other selected from the group consisting of H, C₁₋₃₀-alkyl, C₂₋₃₀-alkenyl, C₂₋₃₀-alkynyl, C₅₋₈-cycloalkyl, C₅₋₈-cycloalkenyl, 5 to 14 membered heterocycloalkyl, 5 to 14 membered heterocycloalkenyl, C₆₋₁₄-aryl and 5 to 14 membered heteroaryl, and
R², R³, R⁴, R⁵, R⁶, R⁷, R⁸, R⁹ and R¹⁰ are independently from each other selected from the group consisting of H, C₁₋₃₀-alkyl, C₂₋₃₀-alkenyl, C₂₋₃₀-alkynyl, C₅₋₈-cycloalkyl, C₅₋₈-cycloalkenyl, 5 to 14 membered heterocycloalkyl, 5 to 14 membered heterocycloalkenyl, C₆₋₁₄-aryl, 5 to 14 membered heteroaryl, halogen, CN, —SCN, NO₂, OH, O—C₁₋₃₀-alkyl, O—C₂₋₃₀-alkenyl, O—C₂₋₃₀-alkynyl, O—C₅₋₈-cycloalkyl, O—C₅₋₈-cycloalkenyl, O-5 to 14 membered heterocycloalkyl, O-5 to 14 membered heterocycloalkenyl, O—C₆₋₁₄-aryl, O-5 to 14 membered heteroaryl, SH, S—C₁₋₃₀-alkyl, S—C₂₋₃₀-alkenyl, S—C₂₋₃₀-alkynyl, S—C₅₋₈-cycloalkyl, S—C₅₋₈-cycloalkenyl, S-5 to 14 membered heterocycloalkyl, S-5 to 14 membered heterocycloalkenyl, S—C₆₋₁₄-aryl, S-5 to 14 membered heteroaryl, C(O)H, CO—C₁₋₃₀-alkyl, CO—C₂₋₃₀-alkenyl, CO—C₂₋₃₀-alkynyl, CO—C₅₋₈-cycloalkyl, CO—C₅₋₈-cycloalkenyl, CO-5 to 14 membered heterocycloalkyl, CO-5 to 14 membered heterocycloalkenyl, CO—C₆₋₁₄-aryl, CO-5 to 14 membered heteroaryl, COOH, NH(C₁₋₃₀-alkyl), N(C₁₋₃₀-alkyl)₂, CONH₂, CONH(C₁₋₃₀-alkyl), CON(C₁₋₃₀-alkyl)₂, SO₂OH, SO₂NH₂, SO₂—C₁₋₃₀-alkyl and SO₂—C₆₋₁₄-aryl,
wherein
C₁₋₃₀-alkyl, C₂₋₃₀-alkenyl and C₂₋₃₀-alkynyl, are optionally substituted with one to nine substituents independently selected from the group consisting of C₅₋₆-cycloalkyl, C₅₋₆-cycloalkenyl, 5 to 10 membered heterocycloalkyl, 5 to 10 membered heterocycloalkenyl, C₆₋₁₀-aryl, 5 to 10 membered heteroaryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂; and one or more CH₂-groups, but not adjacent CH₂-groups of C₁₋₃₀-alkyl, C₂₋₃₀-alkenyl and C₂₋₃₀-alkynyl, and not the CH₂-group directly attached to the core of the compound of formula (1), is optionally replaced by O or S, and
C₅₋₈-cycloalkyl, C₅₋₈-cycloalkenyl, 5 to 14 membered heterocycloalkyl and 5 to 14 membered heterocycloalkenyl are optionally substituted with one to five substituents independently selected from the group consisting of C₁₋₂₀-alkyl, C₂₋₂₀-alkenyl, C₂₋₂₀-alkynyl, C₆₋₁₀-aryl, 5 to 10 membered heteroaryl, OR, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂,
C₆₋₁₄-aryl is optionally substituted with one to five substituents independently selected from the group consisting of C₁₋₂₀-alkyl, C₂₋₂₀-alkenyl, C₂₋₂₀-alkynyl, C₅₋₆-cycloalkyl, C₅₋₆-cycloalkenyl, 5 to 10 membered heterocycloalkyl, 5 to 10 membered heterocycloalkenyl, and 5 to 10 membered heteroaryl OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂,
5 to 14 membered heteroaryl is optionally substituted with one to five substituents independently selected from the group consisting of C₁₋₂₀-alkyl, C₂₋₂₀-alkenyl, C₂₋₂₀-alkynyl, C₅₋₆-cycloalkyl, C₅₋₆-cycloalkenyl, 5 to 10 membered heterocycloalkyl, 5 to 10 membered heterocycloalkenyl, C₆₋₁₀-aryl, OR^{a}, OC(O)—R^{a}, C(O)—OR^{a}, C(O)—R^{a}, NR^{a}R^{b}, NR^{a}[C(O)R^{b}], N[C(O)R^{a}][C(O)R^{b}], halogen, CN and NO₂,
wherein
R^{a} and R^{b} are independently selected from the group consisting of H, C₁₋₂₀-alkyl, C₂₋₂₀-alkenyl and C₂₋₂₀-alkynyl,
C₁₋₂₀-alkyl, C₂₋₂₀-alkenyl and C₂₋₂₀-alkynyl can be substituted with one to five substituents selected from the group consisting of phenyl, OR^{c}, OC(O)—R^{c}, C(O)—OR^{c}, C(O)—R^{c}, NR^{c}R^{d}, NR^{c}[C(O)R^{d}], N[C(O)R^{c}][C(O)R^{d}], halogen, CN and NO₂, and,
C₅₋₆-cycloalkyl, C₅₋₆-cycloalkenyl, 5 to 10 membered heterocycloalkyl, 5 to 10 membered heterocycloalkenyl, C₆₋₁₀-aryl and 5 to 10 membered heteroaryl are optionally substituted with one to five substituents independently selected from the group consisting of C₁₋₁₀-alkyl, C₂₋₁₀-alkenyl, C₂₋₁₀-alkynyl, OR^{c}, OC(O)—R^{c}, C(O)—OR^{c}, C(O)—R^{c}, NR^{c}R^{d}, NR^{c}[C(O)R^{d}], N[C(O)R^{c}][C(O)R^{d}], halogen, CN and NO₂,
wherein
R^{c} and R^{d} are independently selected from the group consisting of H, C₁₋₁₀-alkyl, C₂₋₁₀-alkenyl and C₂₋₁₀-alkynyl,
wherein
C₁₋₁₀-alkyl, C₂₋₁₀-alkenyl and C₂₋₁₀-alkynyl are optionally substituted with one to five substituents selected from the group consisting of halogen, CN and NO₂.

18. An electronic device comprising the compound of claim 11.

19. The electronic device of claim 18, wherein the electronic device is an organic field effect transistor (OFET).

20. A semiconducting material comprising the compound of claim 11.
